(19) United States
(12) Patent Application Publication
(10) Pub. No.: US 2015/0160193 A1
(43) Pub. Date: Jun. 11, 2015

(54) TUMOR CELL ISOLATION/PURIFICATION PROCESS AND METHODS FOR USE THEREOF

(71) Applicant: DIATECH ONCOLOGY, Brentwood, TN (US)

(72) Inventors: Cary Presant, San Marino, CA (US); Mathieu Perree, Montreal (CA); Allan Hallquist, Morning View, KY (US)

(73) Assignee: DIATECH ONCOLOGY, Brentwood, TN (US)

(21) Appl. No.: 14/400,446

(22) PCT Filed: Mar. 14, 2013
(86) PCT No.: PCT/US2013/031300

§ 371 Date: Nov. 11, 2014

Related U.S. Application Data
(60) Provisional application No. 61/647,248, filed on May 15, 2012.

(51) Int. Cl. G01N 33/50; C12N 5/09

(52) U.S. Cl. CPC G01N 33/5011; C12N 5/0693; G01N 2500/10; G01N 2500/04; C12N 2527/00

(57) ABSTRACT

Methods of isolating and purifying hematologic or non-hematologic tumor cells useful in a variety of assays and procedures, including tumor drug efficacy screening such as Microculture Kinetic assays, are disclosed herein. Further, Microculture Kinetic assays and methods suitable for comparing the relative efficacy of generic versus proprietary anti-cancer drugs are also disclosed.

### CROSS-REFERENCE TO RELATED APPLICATIONS

This application is a §371 National Stage Application of PCT/US2013/031300, filed Mar. 14, 2013, which claims priority from U.S. Provisional Patent Application No. 61/647,248, filed on 15 May 2012.

### FIELD OF THE DISCLOSURE

The present disclosure is directed to methods for evaluating the ability of at least one generic and/or proprietary anti-cancer drug candidate to induce apoptosis in cancer cells. More specifically, the present disclosure provides methods that relate to tumor cell purification and isolation, which are particularly optimized for a given specimen's tissue of origin. Further still, the present disclosure provides assays and methodologies, which allow for the accurate and robust comparison of the relative ability of at least one generic and proprietary drug to induce apoptosis in cancer cells.

### BACKGROUND

Cell death may occur in a variety of ways, but most successful anti-cancer drugs tend to cause death of cancer cells by the very specific process of apoptosis. Apoptosis is a mechanism by which a cell disassembles and packages itself for orderly disposal by the body. Apoptosis is commonly used by the body to discard cells when they are no longer needed, are too old, or have become damaged or diseased. In fact, some cells with dangerous mutations that might lead to cancer, and even some early-stage cancerous cells, may undergo apoptosis as a result of natural processes.

During apoptosis, the cell cuts and stores DNA, condenses the nucleus, discards excess water, and undergoes various changes to the cell membrane, such as blebbing, the formation of irregular bulges in the cell membrane. (See FIG. 1.) Apoptosis generally occurs after one of several triggers sends a signal to the cell that it should undergo apoptosis. In many cancer cells, this message system does not work correctly because the cell cannot detect the trigger, fails to send a signal properly after the trigger is received, or fails to act on the signal, or the cell may even have combinations of these problems. The overall effect is a resistance to undergoing apoptosis in some cancer cells.

Cancer, as used herein, includes all cancers or malignancies, both hematologic and non-hematologic, as well as myelodysplastic syndromes (MDS). This contemplates the four major categories for all blood/marrow cancers, solid tumors, and effusions: leukemia, lymphomas, epithelial malignancies, and mesenchymal malignancies.

Although many effective cancer drugs can induce cancerous cells to undergo apoptosis despite their resistance to the apoptotic process, no drug works against all types of cancer cells and no test predicts the relative efficacy of these drugs based on kinetic unit measurements of apoptosis. Accordingly, there is a need to detect whether a particular drug candidate can cause apoptosis in various types of cancer cells and also to determine the drug candidate's effectiveness as compared to other drugs or drug candidates, especially with regard to individual patients.

The Microculture Kinetic Assay (MiCK assay), described in U.S. Pat. No. 6,077,684 and U.S. Pat. No. 6,258,553, is currently used to detect whether leukemia cells from a patient undergo apoptosis in response to a particular drug known to be effective against one or more types of leukemia. In the MiCK assay, cancer cells from a patient are placed in a suspension of a given concentration of single cells or small cell clusters and allowed to adjust to conditions in multiple wells of a microtiter plate. Control solutions or solutions with various concentrations of known anti-cancer drugs, typically those drugs recommended for the patient's cancer type, are introduced into the wells with one test sample per well. The optical density of each well is then measured periodically, typically every few minutes, for a period of hours to days. As a cell undergoes apoptosis-related blebbing, its optical density increases in a detectable and specific fashion. If the cell does not undergo apoptosis or dies from other causes, its optical density does not change in this manner. Thus, if a plot of optical density (OD) v. time for a well yields a straight line curve having a positive slope over the time, followed by a plateau and/or a negative slope, then the anti-cancer drug in that well induces apoptosis of the patient's cancer cells and might be a suitable therapy for that patient. OD v. time data may also be used to calculate kinetic units, the units which can be used to measure apoptosis, which similarly correlate with the suitability of a therapy for the patient. One of ordinary skill in the art will be familiar with the aforementioned general description of the MiCK assay. Further, the contents of U.S. Pat. No. 6,077,684 and U.S. Pat. No. 6,258,553, are herein incorporated by reference in their entirety for all purposes, and provide a more detailed description of the MiCK assay.

Although the MiCK assay has been used to detect the effects of known anticancer drugs on a particular patient's leukemia cancer cells, there remains a need to develop variations of the assay that are specifically adapted to various tumor cell specimen origins. The previously referenced MiCK assay only contemplated blood cancers and specifically Leukemia. Because of the limited scope of current MiCK assays, there is a need in the art for MiCK assays that are particularly suited and sensitive to the detection of apoptosis-related cell/chemical interactions, as encountered in specimens resulting from not only blood cancers, but also other tumor sources. The development of improved MiCK assays and methodologies that are customized for a specimen of a particular origin will enable researchers to provide further accuracy and robustness to the individualized treatment protocols obtainable with the use of MiCK assays. Furthermore, a critical aspect of any screening assay is isolating the cancer cells from other non-cancer cells and materials in a specimen and the purity of the cells on which compounds or drugs are tested.

There is also a great need in the art to develop MiCK assays that are suitable for comparative analysis between proprietary pharmaceutical chemotherapy drugs and their generic equivalents. The term “proprietary” includes single source drugs and/or brand name drugs or chemicals; the term “generic” includes multisource drugs and/or non-brand name drugs or chemicals. The development of such assays and protocols would enable physicians to make cost-effective pre-treatment decisions based upon the relative response of the proprietary drug versus a generic equivalent. These decisions, whether to use a proprietary drug or generic in the treatment of particular cancers, have huge implications for not only individual patients that are faced with enormous treatment costs, but also for the healthcare industry as a whole.

### SUMMARY

It is therefore an object of the current disclosure to provide improved methods of tumor cell isolation and purification from specimens that are to be used in MiCK assays. Further, improvements to the MiCK assay itself are also disclosed, which enable the creation of a more sensitive and robust assay. These methods and assays allow for a determination of apoptosis in all types of cancer cells and are not limited to leukemia.

Methods according to aspects of the present invention are much improved over the MiCK assay protocols heretofore known and provide practitioners with the ability to customize tumor cell purification and isolation protocols depending upon the tumor cell's origin.

The improvements to the MiCK assay include, for example, a refinement to the calculation and derivation of KU values and the coefficient used in determining said KU value. This improvement allows practitioner's to tailor a plan of chemotherapy to a particular patient's disease, by utilizing the disclosed method of deriving more sensitive coefficient and KU values.

It will be readily appreciated that the methodologies disclosed in the present application allow for a more robust and accurate MiCK assay. The improvements to the MiCK assay protocols from the disclosed methodologies lead to corresponding increases in the assay's ability to provide medical practitioners with valuable data to assist in developing patient treatment strategies. Because chemotherapeutic drugs produce significant side effects—regardless of whether they are effective against the type of cancer being treated—those of ordinary skill in the art recognize that it is imperative that the chemotherapeutic drug(s) that are most effective against an individual patient's cancer be identified before initiating treatment. Lacking, however, is an effective and reliable method for achieving this goal.

It is a further object of the current disclosure to provide MiCK assays and methods that are able to compare the relative effectiveness of proprietary versus generic chemotherapy drugs. The ability to compare the relative ability of proprietary versus generic drugs of interest to induce apoptosis in a particular cancer type is an invaluable improvement to the state of the art. Practitioners armed with the ability to choose between generics and proprietary drug choices based upon demonstrated results, from the assays and methods disclosed herein, will be well suited to provide the best treatment strategies for their patients. These micro-scale efficiencies in patient treatment are parallel to the macro-scale efficiencies that will inure to the entire healthcare industry as a whole. The present disclosure allows for huge potential cost savings to the entire healthcare industry because doctors will be enabled by the present methods to choose between generic chemotherapy drugs and proprietary drugs to identify the most effective ones based upon individualized patient MiCK assay results, rather than commercial influences or inconclusive peer-reviewed literature.

In an embodiment, the materials and methods of the present invention are for use in immunological procedures for the isolation and purification (and also enrichment) of tumor cells derived from solid tumor, blood, bone marrow, and effusion specimens. The ability to obtain uncontaminated cancer cell samples is one of the major bottlenecks in the study of tumor development, cancer biology, and drug screening. Tumor biopsies from cancer patients and animal tumor models often contain a heterogeneous population of cells that include normal tissue, blood, and cancer cells. This mixed population makes diagnosis and valid experimental conclusions difficult to obtain and interpret. The present methods alleviate these problems by providing specific protocols tailored to the individual tissue samples' physiological origin.

Another embodiment of the present invention relates to a method of tumor cell isolation and purification comprising the steps of: a) obtaining a tumor specimen; b) treating the specimen with an antibiotic mixture within 24-48 hours; c) mincing, digesting, and filtering the specimen; d) optionally removing non-viable cells by density gradient centrifugation; e) incubating the cell suspension to remove macrophages by adherence; f) performing positive, negative, and/or depletion isolation to isolate the cells of interest; g) removing any remaining macrophages, if necessary, using CD14 antibody conjugated magnetic beads; h) plating the final suspension (e.g., adding the final suspension to the wells of a 384 well plate); and i) incubating plate overnight at 37° C. in a 5% carbon dioxide (CO₂) humidified atmosphere.

Therefore, in an embodiment, the present methods relate to: A method of evaluating the relative apoptosis-inducing activity of an anti-cancer drug candidate, comprising:
a) obtaining cancer cells from a tumor specimen;
b) mincing, digesting, and filtering the specimen;
c) optionally removing non-viable cells by density gradient centrifugation;
d) incubating the cell suspension to remove macrophages by adherence;
e) performing positive, negative, and/or depletion isolation to isolate the cells of interest;
f) removing any remaining macrophages, if necessary, using CD14 antibody conjugated magnetic beads;
g) plating the final suspension;
h) incubating the plate;
i) exposing at least one well of a plated final suspension to at least one first anti-cancer drug candidate or mixtures of the first candidate and other substances;
j) exposing at least one well of a plated final suspension to at least one second anti-cancer drug candidate or mixtures of the second candidate and other substances;
k) measuring the optical density of the wells exposed to the at least one first and second anti-cancer drug candidates, or wells containing mixtures of at least one first or at least one second anti-cancer drug candidate and other substances, wherein said measuring of the optical density occurs in a serial manner at selected time intervals for a selected duration of time;
l) determining a kinetic units value for the at least one first and second anti-cancer drug candidates from the optical density and time measurements;
m) correlating the kinetic units value for each drug candidate with:
   a) an ability of the anti-cancer drug candidate to induce apoptosis in the cancer cells if the kinetic units value is greater than a predetermined threshold;
   b) an inability of the anti-cancer drug candidate to induce apoptosis in the cancer cells if the kinetic units value is less than a predetermined threshold;
n) comparing the determined kinetics units value for each drug candidate; and
o) determining a drug candidate that has a greater relative ability to induce apoptosis in a cancer cell based upon the comparison in step (n).

An embodiment of the invention may also involve the aforementioned steps a)-o), wherein the at least one first and second anti-cancer drug candidates comprise at least one generic drug candidate and one proprietary drug candidate.

The invention also comprises embodiments in which there is a step p) comprising determining the monetary consequences resultant from choosing either the generic or proprietary drug candidate, wherein the drug candidate with the highest relative kinetic units value is selected. In certain embodiments, the monetary consequences are determined based upon treating a single patient with the selected drug with the higher kinetic units value versus the cost that would have occurred based upon the drug candidate with the lower kinetic units value. Generic drugs are generally defined as drugs obtainable from multiple manufacturer sources; whereas, proprietary drugs are defined as those drugs obtainable from only one manufacturer.

Still further embodiments of the present invention comprise a step q) that involves extrapolating the monetary consequences determined from step p) to a target population. Such a target population could comprise any population that is at least 2 patients. Particularly, embodiments of the invention relate to populations that are on a community scale (2 to 10 people, 10 to 20 people, 20 to 50 people, 50 to 100 people, 100 to 300 people, 300 to 1000 people for example), a regional scale (1000 to 2000 people, 2000 to 10000 people for example), a statewide scale (10,000 to 20, 000 people, 20,000 to 50, 000 people for example, or defined as the number of people within a state that are potential candidates for the examined drug treatment), and a nationwide scale (defined as all people within a country that are potential candidates for the examined drug). In a particular embodiment of the invention the target population is a nationwide population from the United States. Such extrapolation may be performed with a suitably programmed computer.

Methods of the present invention may utilize tumor specimens from a variety of sources, for example: solid tumor specimens, blood specimens, bone marrow specimens, and effusion derived specimens are just a few of the specific tumor specimen types suitable for the currently disclosed methods.

Embodiments of the present invention may be utilized to test a wide variety of malignancies. For example, the present disclosure can be used to test the following carcinomas:
Ovarian carcinoma (serous cystadenocarcinoma, mucinous cystadenocarcinoma, endometrioid carcinoma), Ovarian granulosa cell tumor, Fallopian tube adenocarcinoma, Peritoneal carcinoma, Uterine (endometrial) adenocarcinoma, sarcomatoid carcinoma, Cervical squamous cell carcinoma, Endocervical adenocarcinoma, Vulvar carcinoma, Breast carcinoma, primary and metastatic (ductal carcinoma, mucinous carcinoma, lobular carcinoma, malignant phyllodes tumor), Head and neck carcinoma, Oral cavity carcinoma including tongue, primary and metastatic, Esophageal carcinoma, squamous cell carcinoma and adenocarcinoma, Gastric adenocarcinoma, malignant lymphoma, GIST, Primary small bowel carcinoma, Colonic adenocarcinoma, primary and metastatic (adenocarcinoma, mucinous carcinoma, large cell neuroendocrine carcinoma, colloid carcinoma), Appendiceal adenocarcinoma, Colorectal carcinoma, Rectal carcinoma, Anal carcinoma (squamous, basaloid), Carcinoid tumors, primary and metastatic (appendix, small bowel, colon), Pancreatic carcinoma, Liver carcinoma (hepatocellular carcinoma, cholangiocarcinoma), Metastatic carcinoma to the liver, Lung cancer, primary and metastatic (squamous cell, adenocarcinoma, adenosquamous carcinoma, giant cell carcinoma, nonsmall cell carcinoma, NSCLC, small cell carcinoma neuroendocrine carcinoma, large cell carcinoma, bronchoalveolar carcinoma), Renal cell (kidney) carcinoma, primary and metastaic, Urinary bladder carcinoma, primary and metastatic, Prostatic adenocarcinoma, primary and metastatic, Brain tumors, primary and metastatic (glioblastoma, multiforme, cerebral neuroectodermal malignant tumor, neuroectodermal tumor, oligodendroglioma, malignant astrocytoma), Skin tumors (malignant melanoma, sebaceous cell carcinoma), Thyroid carcinoma (papillary and follicular), Thymic carcinoma, Shenoidal carcinoma, Carcinoma of unknown Primary, Neuroendocrine carcinoma, Testicular malignancies (seminoma, embryonal carcinoma, malignant mixed tumors), and others.

The present disclosure can be used to test the following malignant lymphomas, for example: Large cell malignant lymphoma, Small cell lymphoma, Mixed large and small cell lymphoma, Malt lymphoma, Non Hodgkins malignant lymphoma, T cell malignant lymphoma, chronic myelogenous (or myeloid) leukemia (CML), myeloma, other leukemias, mesothelioma, mantle cell lymphomas, marginal cell lymphomas, lymphomas not otherwise specified as to type, and others.

Further still the present disclosure may be utilized to test the following leukemias, for example: AML-acute myelogenous leukemia, ALL-acute lymphoblastic leukemia, Chronic lymphocytic leukemia, Multiple myeloma, Myelodysplastic syndromes-MDS, MDS with myelofibrosis, Waldenstrom's macroglobulinemia, and others.

Also, sarcomas such as the following may be tested with the present disclosure: Leimyosarcoma (uterine sarcoma), GIST-gastrointestinal stromal tumor, primary and metastatic (stomach, small bowel, Colon), Liposarcoma, Myxoid sarcoma, Chondrosarcoma, Osteosarcoma, Ewings sarcoma/PNET, Neuroblastoma, Malignant peripheral nerve sheath tumor, Spindle cell carcinoma, Embryonal rhabdomyosarcoma, Mesothelioma, and others.

Thus, it can easily be recognized that the presently disclosed MiCK assays and methodology represent a dramatic improvement over the MiCK assay previously known in the art, which were merely directed toward Leukemia.

In another embodiment, the present methods relate to: A method of evaluating the ability of an anti-cancer drug candidate to induce apoptosis in a cancer cell line derived from a tumor specimen, comprising:
a) obtaining a tumor specimen;
b) mincing, digesting, and filtering the specimen;
c) optionally removing non-viable cells by density gradient centrifugation;
d) incubating the cell suspension to remove macrophages by adherence;
e) performing positive, negative, and/or depletion isolation to isolate the cells of interest;
f) removing any remaining macrophages, if necessary, using CD14 antibody conjugated magnetic beads;
g) plating the final suspension;
h) incubating the plate;
i) exposing at least one well of a plated final suspension to at least one anti-cancer drug candidate or mixtures of the candidate and other substances;
j) measuring the optical density of the wells exposed to the at least one anti-cancer drug candidate, or wells containing mixtures of at least one anti-cancer drug candidate and other substances, wherein said measuring of the optical density occurs in a serial manner at selected time intervals for a selected duration of time;
k) determining a kinetic units value for the at least one anti-cancer drug candidate from the optical density and time measurements; and
l) correlating the kinetic units value for each drug candidate with:
   a) an ability of the anti-cancer drug candidate to induce apoptosis in the cancer cells if the kinetic units value is greater than a predetermined threshold;
   b) an inability of the anti-cancer drug candidate to induce apoptosis in the cancer cells if the kinetic units value is less than a predetermined threshold.

In some embodiments, each well of the plate comprises a different anti-cancer drug candidate. Further, the method also contemplates embodiments in which a different concentration of the anti-cancer drug candidate is contained in each well. Therefore, the present disclosure may relate to high-throughput assays by which multiple potential drug candidates at multiple potential concentration strengths may be simultaneously tested. This high-throughput ability of embodiments of the present invention are a significant advantage over single drug candidate testing and offers the promise of decreased test cost and increased time savings.

The potential anti-cancer drug candidate concentration which may be loaded into each well of the assay will vary depending upon the manufacturer's recommended dosage and the corresponding dilutions required to achieve the concentration in the well that would correspond to this dosage. For example, the target drug concentration in each well is determined by molarity and can range from 0.01 to 10,000 μM, or 0.001 to 100,000 μM, or 0.1 to 10,000 μM for example, but could also deviate from these disclosed example ranges or comprise any integer contained within these ranges. One skilled in the art will understand how to achieve a target drug concentration by utilizing the manufacturer's recommended blood level concentrations, which may vary plus or minus one serial dilution if enough specimen cells are present.

Embodiments of the invention are able to test all manner of anti-cancer drug candidates. For example, the following anti-cancer drug candidates can be tested by the disclosed methods: Abraxane, Alimta, Amsacrine, Asparaginase, BCNU, Bendamustine, Bleomycin, Caelyx (Doxil), Carboplatin, Carmustine, CCNU, Chlorambucil, Cisplatin, Cladribine, Clofarabine, Cytarabine, Cytoxan (4HC), Dacarbazine, Dactinomycin, Dasatinib, Daunorubicin, Decitabine, Dexamethasone, Doxorubicin, Epirubicin, Estramustine, Etoposide, Fludarabine, 5-Fluorouracil, Gemcitabine, Gleevec (imatinib), Hexamethylmelamine, Hydroxyurea, Idarubicin, Ifosfamide (4HI), Interferon-2a, Irinotecan, Ixabepilone, Melphalan, Mercaptopurine, Methotrexate, Mitomycin, Mitoxantrone, Nitrogen Mustard, Oxaliplatin, Pentostatin, Sorafenib, Streptozocin, Sunitinib, Tarceva, Taxol, Taxotere, Temozolomide, Temsirolimus, Thalidomide, Thioguanine, Topotecan, Tretinoin, Velcade, Vidaza, Vinblastine, Vincristine, Vinorelbine, Vorinostat, Xeloda (5DFUR), Everolimus, Lapatinib, Lenalidomide, Rapamycin, and Votrient (Pazopanib).

However, many other anti-cancer drug candidates, including but not limited to other nonchemotherapy drugs and/or chemicals which can produce apoptosis or which are examined for their ability to produce apoptosis, are also able to be tested by the disclosed methods. Further still, the methods of the present invention are not strictly applicable to anti-cancer drug candidates, but rather embodiments of the disclosed methods can be utilized to test any number of potential drug candidates for a whole host of diseases.

### BRIEF DESCRIPTION OF THE DRAWINGS

These and other features, aspects, and advantages of embodiments of the present invention will become better understood with regard to the following description, appended claims, and accompanying drawings, where:

FIG. 1: shows a time sequenced photomicrograph of a cancer cell moving through the stages of apoptosis. The first panel on the left (1) shows the cell prior to apoptosis. The middle panel (2) shows the cell during apoptosis and blebbing is apparent. The last panel on the right (3) shows the cell after apoptosis is complete or nearly complete.

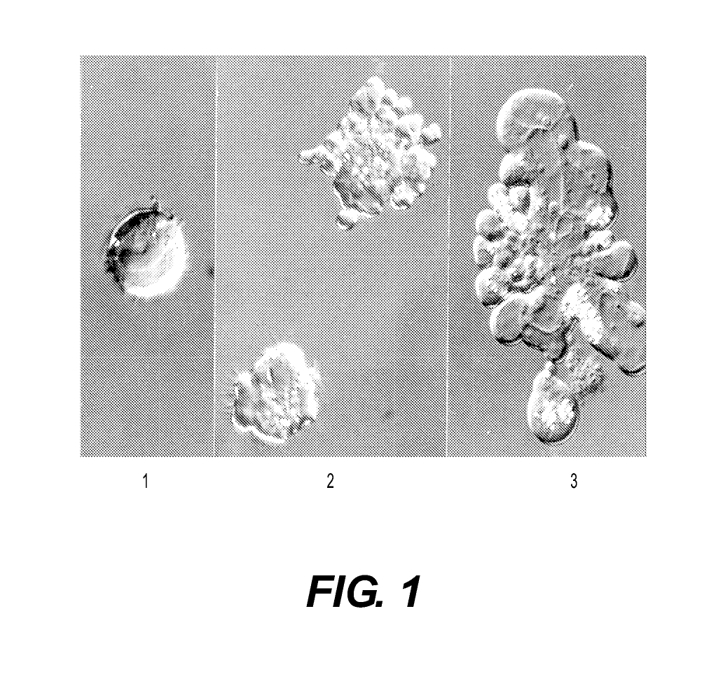

FIG. 2: shows the overall survival of patients. Red line, patients whose therapy was based on using the MiCK assay results. Blue line, patients whose therapy was not based on using the MiCK assay results. Cross hatches in curves indicate patients censored. Small numbers above the abscissa indicate patients at risk at each time point. By log rank analysis the curves are statistically different p=0.04.

FIG. 3: shows relapse-free interval in patients. Red line, patients whose therapy was based on using the MiCK assay results. Blue line, patients whose therapy was not based on using the MiCK assay results. Cross hatches in curves indicate patients censored. Small numbers above the abscissa indicate patients at risk at each time point. By log rank analysis the curves are statistically different p<0.01.

FIG. 4: shows a comparison between breast and lung specimens and illustrates whether there is a difference between the tissue specimen types with relation to whether generics or proprietary drugs are more effective in one type versus the other. Note: For breast cancer only single drugs were used to ID generic and proprietary while for lung and colon multiple drugs were considered. The chi-square (χ²) analysis shows that the % g≧p for breast (97.7%) is not statistically significantly different than the % for lung (93.8%) using Fisher's exact test (p-value=0.57).

FIG. 5: shows a comparison between breast and colon specimens and illustrates whether there is a difference between the tissue specimen types with relation to whether generics or proprietary drugs are more effective in one type versus the other. The chi-square analysis shows that the % g≧p for breast (97.7%) is statistically significantly different than the % for colon (71.4%) using Fisher's exact test (p-value<0.05).

FIG. 6: shows a comparison between breast and colon+lung specimens and illustrates whether there is a difference between the tissue specimen types with relation to whether generics or proprietary drugs are more effective in one type versus the other. The chi-square analysis shows that the % g≧p for breast (97.7%) is not statistically significantly different than the % for colon+lung (89.7%) using Fisher's exact test (p-value=0.19).

FIG. 7: shows a comparison between colon and lung specimens and illustrates whether there is a difference between the tissue specimen types with relation to whether generics or proprietary drugs are more effective in one type versus the other. The distributions of lung to colon for best proprietary (p=0.16) and best generic (p=0.45) shows that there is insufficient evidence to conclude lung and colon differ. The non-parametric Wilcoxon test was used due to small sample size with the colon group.

FIG. 8: shows a photomicrograph of cells in a well plate before overnight incubation.

FIG. 9: shows a photomicrograph of cells in a well plate after a 15 hour incubation.

FIG. 10: shows the apoptotic response of cancer cells to the 37 tested anti-cancer drug candidates at various concentrations.

### DETAILED DESCRIPTION

### General MiCK Assay Protocol

The disclosure relates to evaluation of anti-cancer drug candidates' effectiveness in causing apoptosis in cancer cells using a spectrophotometric assay to measure optical density (OD) over a period of time. In one embodiment, the disclosure includes a method of evaluating such anti-cancer drug candidates by applying the drug candidates to cancer cells in an assay similar to the Microculture Kinetic (MiCK) assay as disclosed in U.S. Pat. Nos. 6,077,684 and 6,258,553, previously referenced, and both incorporated by reference in their entireties.

According to one specific embodiment, the assay may proceed by selecting an anti-cancer drug candidate and selecting at least one cancer cell, derived from an obtained tumor specimen, on which to test the drug.

In one embodiment, the cancer cells may be suspended as a single-cell suspension in culture medium, such as RPMI. As used herein, a “single cell suspension” is a suspension of one or more cells in a liquid in which the cells are separated as individuals or in clumps of 10 cells or fewer. The culture medium may contain other components, such as fetal-bovine serum or components specifically required by the cancer cells. These components may be limited to those necessary to sustain the cells for the duration of the assay, typically at least 24 hours and not longer than 120 hours.

Suspended cells may be tested by placing samples in wells of a spectrophotometric plate. The cells may be suspended at any concentration such that during the spectrophotometric measurements of Optical Density (OD), the beam of the plate reader normally passes through only one cell layer at a time. For most cells a concentration of between 2×10⁵ and 1×10⁶ cells/mL may be used. Concentration may be increased for small cells and decreased for large cells. To more precisely determine the appropriate cell concentration, the volume of cell suspension to be used in drug candidate test samples may be added to at least one concentration test well of the plate. If the well will be prefilled with additional medium during testing of the drug candidates, then the concentration test well may similarly be prefilled with additional medium. After the concentration test well is filled, the plate may be centrifuged (e.g. for 30-120 seconds at 500 RPM) to settle the cells on the bottom of the well. If the cell concentration is appropriate for the assay, the cells should form a monolayer without overlapping. Cell concentration may be adjusted as appropriate until this result is achieved. Multiple concentrations of cells may be tested at one time using different concentration test wells.

According to embodiments where the cells may grow significantly overnight or during another period of time between placement of the cells in the plate and commencement of the drug candidate assay, the cell concentration may be adjusted to initially achieve less than a monolayer to allow for growth such that sufficient cells for a monolayer will be present when the drug candidate assay commences.

After the appropriate cell concentration has been determined, the drug candidate assay may proceed by filling test and control wells in the plate with an appropriate volume of medium and an appropriate number of cells. In other embodiments the well may be partially pre-filled with medium alone.

After filling, the cells may be allowed to adjust to the plate conditions for a set period of time, such as at least 12 hours, at least 16 hours, at least 24 hours, or 12-16 hours, 12-24 hours, or 16-24 hours. An adjustment period may be omitted for certain cell types, such as leukemia/lymphoma cell lines or other cell types normally present as individual cells. The adjustment period is typically short enough such that the cells do not experience significant growth during the time. The adjustment period may vary depending on the type of cancer cells used in the drug candidate assay. Adjustment may take place under conditions suitable to keep the cells alive and healthy. For example, the plate may be placed in a humidified incubator at 37° C. under 5% CO₂ atmosphere. For some cell types, particularly cell types that do not undergo an adjustment period, such as leukemia or lymphoma cell lines, the plate may be centrifuged (e.g. for 2 minutes at 500 RPM) to settle the cells on the bottom of the wells.

The drug candidate and any control drugs or other control samples may be added to the wells after the adjustment period. Typically the drug candidate will be added in a small volume of medium or other liquid as compared to the total volume of liquid in the well. For example, the volume of drug added may be less than 10% of the total volume of liquid in the well. Drug candidates may be added in multiple dilutions to allow determination of any concentration effects. Although many drug candidates may be water-soluble, drug candidates that are not readily soluble in water may also be tested. Such candidates may be mixed with any appropriate carrier. Such candidates may preferably be mixed with carriers anticipated for actual clinical use. Viscous drug candidates may require substantial dilution in order to be tested. Drug candidates with a strong color may benefit from monitoring of OD in test wells containing only the drug candidate and subtraction of this OD from measurements for the test sample wells.

After addition of the drug candidate, the cells may be allowed another short period of adjustment, for example of 15 minutes or 30 minutes. The cells may be placed under conditions suitable to keep the cells alive and healthy. For example, the plate may be placed in a humidified incubator at 37° C. under 5% CO₂ atmosphere. After this short adjustment period, a layer of mineral oil may be placed on top of each well to maintain CO₂ in the medium and prevent evaporation.

The plate may then be placed in a spectrophotometer configured to measure the OD at a defined wavelength. The spectrophotometer may be configured to measure OD at a wavelength, for example, of from 550 to 650 nm, or 600 to 650 nm, or more particularly the spectrophotometer is configured to read the OD at a wavelength of 600 nm, for each well at a given time interval for a given total period of time. For example, OD for each well may be measured periodically (i.e. serially) over a time frame of seconds, minutes, or hours, for a period of from approximately 24 hours to 120 hours, approximately 24 hours to 72 hours, or approximately 24 hours to 48 hours. Or, for certain cells, measurements for a period of as little as 12 hours may be sufficient. In specific embodiments, measurements may be taken every 5 to 10 minutes. The spectrophotometer may have an incubated chamber to avoid spontaneous death of the cells.

Spectrophotometric data may be converted to kinetic units. Kinetic units are determined by the slope of the curve created when the change in the OD at the measured wavelength, for example 600 nm, caused by cell blebbing, is plotted as a function of time. Specific information regarding the calculation of kinetic units is provided in Kravtsov, Vladimir D. et al., Use of the Microculture Kinetic Assay of Apoptosis to Determine Chemosensitivities of Leukemias, Blood 92:968-980 (1998), herein incorporated by reference in its entirety for all purposes. Kinetic unit determination is also discussed in more detail below. The Optical density for a given drug candidate at a given concentration may be plotted against time. This plot gives a distinctive increasing curve if the cells are undergoing apoptosis. In comparison, if the drug candidate has no effect on the cells (e.g. they are resistant), then the curve is similar to that obtained for a control sample with no drug or drug candidate. Cell death due to reasons other than apoptosis can also be determined by the current assay and is useful in eliminating false positives from drug candidate screening. For example, cell necrosis produces a distinctive downward sloping curve easily distinguishable from the apoptosis-related curve. Further, general cell death also causes a downward curve.

### Kinetic Units of Apoptosis (KU)

The effectiveness of a drug candidate may be determined by the value of the kinetic units it produces in a modified MiCK assay. The KU is a calculated value for quantifying apoptosis. Kinetic units may be determined as follows:

Apoptosis(KU)=(Vmax_{Drug Candidate Treated}−Vmax_{Control})×60×X/(OD_{control}−OD_{blank})

The KU is a calculated value for quantifying apoptosis. The optical densities (OD) from each well are plotted against time. The maximum slope of the apoptotic curve (Vmax) is calculated for each plot of drug treated microculture. It is then compared to the Vmax of a control well without drug (calculated at the same time as the Vmax of the drug exposed cells). For convenience, the Vmax is multiplied by 60 to convert the units from mOD/minute to mOD/hour. The data are normalized with a coefficient (coefficient=X/(OD_{control}−OD_{blank}), which is discussed below.

### Coefficient

As stated above, the coefficient is a calculated value for normalizing the amount of cells per well when measuring apoptosis and quantifying said apoptosis in Kinetic Units.

The coefficient is calculated as follows:

Coefficient: X/(OD_{control}−OD_{blank})

X=optimal optical density value for the cell type tested (determined empirically)

OD_{control}=average optical density of all the control wells

OD_{blank}=average optical density of all the blank wells

A coefficient of 1.000 means that the cell concentration in the well is optimal. A coefficient value below 1.000 means that the cell concentration is higher than the optimal concentration. If the coefficient value is above 1.000, it means that the cell concentration in the well is suboptimal. The acceptable coefficient values for an optimal MiCK assay are between 0.8 and 1.5. If the value is under 0.8, the coefficient will erroneously reduce the value of the calculated KU. If the value is above 1.5, there will not be enough cells per well to detect the signal of apoptosis. The “X” in the formula will vary depending on the cell type. For solid tumor specimens, this value is 0.09. For most of the leukemias, the value is 0.15. For CLL (chronic lymphocytic leukemias) and the lymphomas, the value is 0.21.

This “X” value is adapted to the tumor type and determined empirically. Thus, the coefficient is developed by trial and error, using different concentrations of cells and by checking them under a microscope while looking for complete proper coverage in the well. The proper well is read by a reader and the OD becomes the new X value. Further information regarding this equation may be found in Kravtsov et al. (Blood, 92:968-980), which was previously incorporated herein by reference.

In addition to allowing determinations of whether or not a drug candidate causes apoptosis, kinetic unit values generated using the current assay may be compared to determine if a particular drug candidate performs better than or similar to current drugs. Comparison of different concentrations of a drug candidate may also be performed and may give general indications of appropriate dosage. Occasionally some drugs may perform less well at higher concentrations than lower concentrations in some cancers. Comparison of kinetic unit values for different concentrations of drug candidates may identify drug candidates with a similar profile.

Overall, evaluation of an anti-cancer drug candidate may include any determination of the effects of that drug candidate on apoptosis of a cancer cell. Effects may include, but are not limited to induction of apoptosis, degree of induction of apoptosis as compared to known cancer drugs, degree of induction of apoptosis at different drug candidate concentrations, and failure to induce apoptosis. The anti-cancer drug evaluation assay may also be able to detect non-drug-related or non-apoptotic events in the cancer cells, such as cancer cell growth during the assay or cell necrosis.

Any statistically significant positive kinetic unit value may indicate some tendency of a drug candidate to induce apoptosis of a cancer cell. For many clinical purposes, however, drug candidates or concentrations of drugs only able to induce very low levels of apoptosis are not of interest. Accordingly, in certain embodiments of the disclosure, threshold kinetic unit values may be set to distinguish drug candidates able to induce clinically relevant levels of apoptosis in cancer cells. For example, the threshold amount may be 1.5, 2 or 3 kinetic units. The actual threshold selected for a particular drug candidate or concentration of drug candidate may depend on a number of factors. For example, a lower threshold, such as 1.5 or 2, may be acceptable for a drug candidate able to induce apoptosis in cancer types that do not respond to other drugs or respond only to drugs with significant negative side effects. A lower threshold may also be acceptable for drug candidates that exhibit decreased efficacy at higher concentrations or which themselves are likely to have significant negative side effects. A higher threshold, such as 3, may be acceptable for drug candidates able to induce apoptosis in cancer types for which there are already suitable treatments.

In another embodiment the following threshold ranges can be utilized:
0-1 KU: non-sensitive
1-2 KU: low sensitivity
2-3 KU: low/moderate sensitivity
3-5: KU: moderate sensitivity
>5 KU: sensitive
   Preferably, the following threshold ranges can be utilized:
0-1 KU: non-sensitive
1-2.6 KU: low sensitivity;
2.6-4.2 KU: low/moderate sensitivity
4.2-5.8: KU: moderate sensitivity
>5.8 KU: sensitive.
   Preferably, the KU value is ≧7, more preferably the KU value is ≧8, even more preferably the KU value is ≧9, and most preferably the KU value is ≧10.

These ranges were established based on a statistical analysis of cancer cells. The ranges establish a baseline for relative comparison of chemotherapeutic drugs being tested on a specific cell type. Test outcomes may be affected by extenuating factors such as:
time elapsed from obtaining sample to testing,
quantity of viable cells available to test,
microbial contamination of specimen,
quality or viability of cells being tested,
cell type, and
recent treatment such as chemotherapy or radiation therapy

These factors suggest some elasticity in the predictive values of the kinetic response reported. Clinical sensitivity to chemotherapy drugs is not completely limited to outcomes as forecast in the above ranges. The KU measurement of drug-induced apoptosis in the assay may be used by physicians to develop an individual patient treatment regimen along with other important factors such as; patient history, prior treatment results, overall patient health, patient comorbidities, patient preferences, as well as other clinical factors.

Therefore, the particular ranges of KU value utilized will be dependent upon context. That is, depending upon the particular type of tumor cell being tested, the particular drug being utilized, and the particular patient or patient population under analysis. The KU value therefore represents a dependable and flexible analytical variable that can be tailored by the practitioner of the disclosed methods to create a suitable metric by which to evaluate a given drug's effect.

### Drug Candidates

According to a specific embodiment, the anti-cancer drug candidates may be any chemical, chemicals, compound, compounds, composition, or compositions to be evaluated for the ability to induce apoptosis in cancer cells. These candidates may include various chemical or biological entities such as chemotherapeutics, other small molecules, protein or peptide-based drug candidates, including antibodies or antibody fragments linked to a chemotherapeutic molecule, nucleic acid-based therapies, other biologics, nanoparticle-based candidates, and the like. Drug candidates may be in the same chemical families as existing drugs, or they may be new chemical or biological entities.

Drug candidates are not confined to single chemical, biological or other entities. They may include combinations of different chemical or biological entities, for example proposed combination therapies. Further, although many examples herein relate to an assay in which a single drug candidate is applied, assays may also be conducted for multiple drug candidates in combination. It is also important to understand that embodiments of the invention may utilize the metabolites of the various drug candidates in a method as described.

More than one drug candidate, concentration of drug candidate, or combination of drugs or drug candidates may be evaluated in a single assay using a single plate. Different test samples may be placed in different wells. The concentration of the drug candidate tested may be, in particular embodiments, any concentration in the range from 0.1 to 10,000 μM, or any concentration in the range from 0.01 to 10,000 μM, or any concentration in the range from 0.001 to 100,000 μM, for example. The concentration tested may vary by drug type, and the aforementioned example concentrations are not to be considered as limiting, for the skilled artisan will understand how to construct the appropriate concentration for utilization with the taught methods and assays, depending upon the particular anti-cancer drug tested.

### Plate and Spectrophotometer Systems

In specific embodiments, the plate and spectrophotometer may be selected such that the spectrophotometer may read the plate. For example, when using older spectrophotometers, one may use plates with larger wells because the equipment is unable to read smaller-well plates. Newer spectrophotometers may be able to read a plate with smaller wells. In one embodiment, the diameter of the bottom of each well is no smaller than the diameter of the light beam of the spectrophotometer. In a more specific embodiment, the diameter of the bottom of each well is no more than twice the diameter of the light beam of the spectrophotometer. This helps ensure that the OD at the measured wavelength, 600 nm for example, of a representative portion of the cells in each well is accurately read. The spectrophotometer may make measurement at wavelengths other than 600 nm. For example, the wavelength may be +/−5 or +/−10. However, other wavelengths may be selected so as to be able to distinguish blebbing.

Spectrophotometers may include one or more computers or programs to operate the equipment or to record the results. In one embodiment, the spectrophotometer may be functionally connected to one or more computers able to control the measurement process, record its results, and display or transmit graphs plotting the optical densities as a function of time for each well.

Plates designed for tissue culture may be used, or other plates may be sterilized and treated to make them compatible with tissue culture. Plates that allow cells to congregate in areas not accessible to the spectrophotometer, such as in corners, may work less well than plates that avoid such congregation. Alternatively, more cancer cells may be added to these plates to ensure the presence of a monolayer accessible to the spectrophotometer during the assay. Plates with narrow bottoms, such as the Corning Costar® half area 96 well plate, may also assist in encouraging formation of a monolayer at the bottom of the well without requiring inconveniently low sample volumes. Other plates, such as other 96-well plates or smaller well plates, such as 384-well plates, may also be used.

### Modified MiCK Assay Protocol

There are a number of distinctions between the MiCK assay protocol previously described in U.S. Pat. No. 6,077,684 and U.S. Pat. No. 6,258,553, and the MiCK assay protocol currently disclosed, for example:
a. overnight incubation for solid tumor sample specimens;
b. low volume wells, since solid tumors give fewer cells than blood samples;
c. the cell concentration is adjusted via visual interpretation;
d. the cell will adhere to the bottom of the wells and spread/stretch overnight;
e. utilization of a special incubation chamber to diffuse heat evenly;
f. avoiding the edges of the plates when one loads the cells into the wells;
g. utilization of an automated pipettor, to plate the cells, media (RPMI+10% Fetal Bovine Serum+Penstrep) and drugs;
h. utilization of proprietary code created to translate template in a format that a robot can understand;
i. cell isolation ends when we have a pure cell suspension ready for plating;
j. a cell count is used to adjust the cell concentration;
k. adjustment of the concentration to 1*10⁶ cells per ml;
l. a test well is done to observe the cell distribution;
m. if the cells are not in good shape, more cells are added to each well;
n. if the test well seems adequate (monolayer of uniformly distributed cells that covers all the area), one proceeds to the next step (plating);
o. if test well not adequate, adjustment of the cell concentration (diluting the cells, or concentrate the cells) and retesting a new well until the cell distribution in the well is satisfactory;
p. at this point (after the aforementioned steps) the stock solution is ready to be plated into additional wells in that plate, until the cells are depleted;
q. using the selected cell concentration, the cell suspension is distributed in the plate into as many wells as possible retaining enough cells to do at least 1 cytospin and ICC (immunocytochemistry) if possible;
r. an automated pipettor is used to distribute the cells while avoiding the edge wells of the plates;
s. the edge wells are filled with media;
t. a configuration file was manufactured to eliminate the bubble problem that was encountered with the automated pipettor (spotting). This feature is important as it eliminates the formation of bubbles in the media during the assay which artificially elevate the slope values which leads to markedly elevated KU values;
u. this plate (that has undergone the aforementioned steps) is now ready for overnight incubation (approximately 15 hour);
v. the incubation allows time for the cells to adhere to the bottom of the wells as well as to metabolically stabilise;
w. after the incubation plate is removed from the incubator, the cell distribution and viability are evaluated from an observation of the plate with an inverted microscope. A photomicrograph of a representative well is taken;
x. the plate is then ready for addition of the drugs (for example possible anti-cancer agents) by the automated pipettor;
y. drugs are selected by the treating oncologist (for example), and NCCN panels, then off panel drugs (off label).
z. an incubation of 30 minutes at 37° C. and 5% CO₂ is done to allow for pH equilibration;
aa. oil is added to every well to prevent air exchange and evaporation;
bb. the plate is placed in a reader and the assay is started;
cc. the assay automatically terminates after 576 reads (48 hours, 5 min intervals); these settings can be adjusted as needed;
dd. the assay can be manually terminated if all the reactions are deemed to have been completed prior to the 48 hours;
ee. the Coefficient may be defined as: X/(OD ctrl−OD blanks) where X is the optimal value of a given cell line. OD is optical density. The coefficient was developed by trial and error, using different concentrations of cells and by checking them under a microscope while looking for complete proper coverage in the well. The proper well was read by a reader and the OD became the new X value;
ff. a trained observer may assess cytologic characteristics of cells at all stages of purification;
gg. a trained observer may analyze ranking of drugs;
hh. a trained observer may analyze best drugs or combinations; and
ii. a trained observer may analyze most active drug candidates (may also include analyzing drug metabolites) and other developed drugs or agents.

The differences over the current state of the art described above are neither taught nor suggested by the prior art, and are not self evident to anyone who practices the art previously disclosed.

Another difference between the original MiCK assay and the current version is that the original MiCK assay avoided adherence of the cells to the plate wells while the current version used adherence to the plate well walls. Adherence of the cells to the well walls is required for cancers and sarcomas that are not of blood or bone marrow origin. Non adherence of the cells to the well walls is required for testing leukemia and lymphomas (cancers of blood or bone marrow origin). The reason for this difference is that leukemia and lymphoma cells will grow in a form of a suspension in vitro. The cells do not require a permanent close contact with each other. At the opposite, cells originating form solid tumor specimens, do require cell to cell contact and attachment to the surface of the well. This will stimulate cell survival and sometimes growth.

Now that a few of the differences between the present disclosure and previous MiCK assay protocols have been generally set-forth, it will be illustrative to provide examples of embodiments of the protocols of the present invention. These Examples are included to describe exemplary embodiments only and should not be interpreted to encompass the entire breadth of the invention.

### Examples

### Correlation of Drug-Induced Apoptosis Assay Results with Oncologist Treatment Decisions and Patient Response and Survival

### Brief Overview of Experimental Protocol and Results

An observational prospective non-blinded clinical trial was performed to determine the effect of drug-induced apoptosis assay results on treatments planned by oncologists. Purified cancer cells from patient biopsies were placed into the Microculture Kinetic (MiCK) assay, a short-term culture, which determined the effects of single drugs or combinations of drugs on tumor cell apoptosis. Oncologist received the assay results prior to finalizing the treatment plan.

Use of a MiCK assay, according to an embodiment of the present invention, was evaluated and correlated with patient outcomes. Results: 44 patients with successful MiCK assays from breast cancer (16), non-small cell lung cancer (6), non-Hodgkin's lymphoma (4), and others were evaluated. 4 patients received adjuvant chemotherapy after MiCK, and 40 received palliative chemotherapy with a median line of therapy of 2. Oncologists used the MiCK assay, of the present disclosure, to determine chemotherapy (users) in 28 (64%) and did not (non-users) in 16 patients (36%). In users receiving palliative chemotherapy, complete plus partial response rate was 44%, compared to 6.7% in non-users (p<0.02). The median overall survival was 10.1 months in users versus 4.1 months in non-users (p=0.02). Relapse-free interval was 8.6 months in users versus 4.0 months in non-users (p<0.01). Conclusions: MiCK assays according to the present invention are frequently used by oncologists. Outcomes appear to be statistically superior when oncologists use chemotherapy based on MiCK assay results of the present invention, as compared to when they do not use the assay results. When available to oncologists, a MiCK assay according to the present invention, and its results help to determine patient treatment plans.

### Specific Experimental Protocol and Detailed Results

An observational non-randomized, multi-institutional prospective trial was conducted in order to determine how often physicians would use the results of the currently disclosed embodiment of the MiCK assay, when the physicians knew the results of the assay prior to planning and initiating chemotherapy.

Patients with cancer of any stage, primary or recurrent, were eligible for the experiment. Sterile Tumor specimens with as much as 1.0 cm³ of viable tumor tissue, or 1000 ml of malignant effusions, or 5 ml of leukemic bone marrow aspirate were taken from patients. The tumor specimens were then subjected to the following experimental protocols.

### Example 1

### Generic Cell Isolation Protocol

Within 24 to 48 hours of collection, the specimen was minced, digested with 0.25% trypsin and 0.08% DNase for 1-2 hours at 37 C.°, and then filtered through a 100 micrometer cell strainer. When necessary, non-viable cells were removed by density gradient centrifugation. The cell suspension was then incubated for 30 min at 37° C. in a tissue culture flask to remove macrophages by adherence. For epithelial tumors lymphocytes were removed by 30 minute incubation with CD2 antibody conjugated magnetic beads for T lymphocytes and CD19 antibody conjugated magnetic beads for B lymphocytes. Remaining macrophages were removed, if necessary, using CD14 antibody conjugated magnetic beads. The final cell suspension was plated into a 96-well half-area plate, 120 microliter aliquot per well. The plate was incubated overnight at 37° C. with 5% carbon dioxide humidified atmosphere. 5×10⁴ to 1.5×10⁵ cells were seeded per well depending on the cell volume to give adequate well-bottom coverage.

Human JURL-MK2 chronic leukemia in blast crisis cell line (DSMZ, Germany) was used as a positive control for MiCK assays performed with patient tumor cells. RPMI-1640 medium without phenol red was used for all cultures. It was supplemented with 10% fetal bovine serum, 100 units/mL of penicillin, and 100 micrograms/mL of streptomycin. Cell counts and viability were evaluated by trypan blue dye exclusion.

Each tumor cell preparation, after purification of contaminating and necrotic cells, was analyzed to confirm the presence of malignancy cytologically. If an adequate number of cells were available, immunocytochemical stains were also performed to better characterize the tumor phenotype. All specimens achieved at least 90% pure tumor cell content by visual estimation by an experienced pathologist and 90% viability by trypan blue exclusion.

The above described generic isolation protocol may be modified by the below described specimen specific isolation protocols.

### Example 2

### Solid Tumor Cell Specific Isolation Protocol

Within 24 to 48 hours of collection, the specimen was treated as follows in order to purify and isolate cells from solid tumors:
Take the specimen out of the transport tube.
Put in a petri dish in 13 ml of PBS+high concentration of antibiotics (200 units/ml Penicillin+200 μg/ml streptomycin) and take measurements and picture of the specimen. The PBS+antibiotics solution is made from solutions mixed together in the lab using proprietary protocols.
Wash 3 times in petri dishes (3 different petri dishes) with 13 ml of PBS+high concentration of antibiotics (200 units/ml Penicillin+200 μg/ml streptomycin.
If contamination is suspected, incubate 20 min in a tube with PBS+high concentration of antibiotics.
Transfer the specimen into another petri dish with 1 to 3 ml (depending on specimen size) of RPMI 50% Fetal Bovine Serum (FBS) for mincing.
1) Next, the specimen was minced, and digested with 0.25% trypsin (enzyme can vary with tissue being used) and 0.08% DNase for 1-2 hours at 37 C.°,
Enzyme will vary with the tumor type following protocols developed by researchers' experience with various tissues.
If contaminating non-tumor tissue is identified in the specimen, remove these parts with scalpels.
Mince in 1 mm pieces with scalpels size 10 or 21.
Collect the pieces with forceps, put in a 15 ml tube+10-12 ml of enzyme (the enzyme depends on the tumor type; see Table 1), incubate 45-60 min in the incubator at 37° C. on a “rotator”.
Wash the petri dish used for mincing with RPMI (4-5 ml), 2-3 times.
Put the washing in a 15 ml tube, let settle 2-3 min
Remove the supernatant and put in a new 15 ml tube, check the viability of cells with the hemacytometer and trypan blue dye (this gives an early indication on how difficult and/or easy the processing should be).
Put the pellet in a 15 ml tube with the enzyme and incubate at 37° C. on the rotator for 45-60 min
After the incubation, collect the supernatant and put back the remaining pieces in fresh enzyme at 37° C. for another 45-60 min
2) Next, the specimen was filtered through a 100 micrometer cell strainer.
Depending on tumor type and amount of “non-cancer cell tissue” remaining, one could also use 40 and 70 μM strainer or filcon.
If the supernatant is viscous or if it contains a lot of debris, it will block the cell strainer. In that case, one may make the determination to do a “pre-filtration” using sterile gauze over a 50 ml tube. Then proceed with the cell strainer filtration process referenced above.
Centrifuge the filtered cell suspension 1500 RPM 5 min
Discard the supernatant. To the pellet, add 5 ml of red blood cell lysis solution (standard NH₄Cl containing lysis solution: Nh₄Cl0.15M+KHCO₃ 10 mM+EDTA-4Na 0.1 mM, pH 7.2), incubate 2-3 min and add 5 ml of RPMI 10% FBS.
Centrifuge 5 min 1500 RPM. Resuspend the pellet in RPMI 10% FBS (1-10 ml depending on the pellet size).
Collect the second fraction in the enzyme and repeat the steps above.
Check the viability of all fractions and pool. Do a cytospin stain with Wright Giemsa to verify the cell content of the population. NOTE: this is done numerous times during the process of purification.
3) When necessary, non-viable cells were removed by density gradient centrifugation.
Density gradient centrifugation (optiprep): first layer=2 ml cells+4.45 ml optiprep 40% in RPMI, second layer=optiprep 22.5% in RPMI, 3^{rd} layer=0.5 ml of RPMI. Centrifuge at 2000 RPM for 20 min
Collect the viable cell layer, add 10 ml of RPMI 10% FBS, centrifuge at 1500 RPM for 5 min
Resuspend the pellet in RPMI 10% FBS (volume depends on the pellet size and on the next step required).
If mucin is present in the specimen: resuspend the pellet in 10 ml of PBS+20 mM DTT and incubate at 4° C. for 30 min to disintegrate the mucin. Wash with RPMI 1500 rpm for 5 min Resuspend the pellet in RPMI 10% FBS.
If the specimen is highly necrotic with presence of debris: Percoll 20% in HBSS, centrifuge at 800×g for 10 min
4) The cell suspension was then incubated for 20 min at 37° C. in a tissue culture flask to remove macrophages by adherence.
The size and quantity of the flask and the volume used depends on the amount of cells. Examples:
   1-5×10⁶ cells: 25 cm² flasks, 3-4 ml each
   1×10⁷ cells: 75 cm² flasks, 8 ml each
   1×10⁸ cells: 175 cm² flasks, 20 ml each
After incubation, collect the cell suspension, wash the flask 3 times with RPMI 10% FBS, pool all the washing fractions, centrifuge 1500 RPM for 5 min
5) For epithelial tumors, lymphocytes were removed by 30 minute incubation with CD2 antibody conjugated magnetic beads for T lymphocytes and CD19 antibody conjugated magnetic beads for B lymphocytes.
Beads to use: T lymphocytes=CD2; B lymphocytes=CD19; neutrophils=CD15; monocytes/macrophages=CD14, all leukocytes=CD45 (use CD45 if there are no clumps).
Macrophages are usually removed by adherence, not with the beads. The reason is that if clumps of tumor cells are present, they can also contain macrophages. If we use beads to remove the macrophages, it could also remove the tumor cells at the same time.
Resuspend the pellet in a small volume of PBS 2% FBS (0.2 to 2 ml).
Wash the beads suspension 3 times with the PBS 2% FBS.
Add the beads to the cell suspension and incubate 30 min at room temperature on the rotator.
Put the tube on the magnet, wait for 1 min
Collect the cell suspension, put in a 15 ml tube with 5 ml of RPMI 10% FBS
Put the tube of the cell suspension on a magnet again to remove remaining beads, collect the cell suspension and put in a new 15 ml tube.
Centrifuge at 1500 RPM for 5 min
Resuspend in RPMI 10% FBS, the volume depends on the pellet size. Do a cell count and determine viability, do a cytospin to determine cell content.
6) Remaining macrophages were removed, if necessary, using CD14 antibody conjugated magnetic beads.
This step would be done at the same time that the other beads are being processed as outlined above in step 5.
Look at the cell viability. An additional step may be required if the viability is less than 80-85%. If that is the case, repeat the density gradient centrifugation (optiprep) as describe on step 3. This will remove the dead cells.
7) The final cell suspension was plated into a 96-well half-area plate, or a 384 well plate with 62.5 microliter aliquot per well, or a 384 well plate with 20 microliter aliquot per well, as indicated in Table 2.
Adjust the cell concentration to 1×10⁶ cells per ml.
Do a test well. For corning 384=15 μl of RPMI 10% FBS+45 μl of cell suspension→centrifuge at 500 rpm for 1 min. For Greiner=2.5 μl or RPMI 10% FBS+15 μl of cell suspension→centrifuge at 500 rpm for 30 sec.
Look at the well under the inverted microscope. The cells should touch each other but not be overlapping. Adjust the cell concentration as needed by concentrating (centrifuge and remove medium) or diluting (adding medium).
Repeat until optimal cell concentration is found.
Put the cells in the well plate.
8) The plate was incubated overnight at 37° C. with 5% carbon dioxide humidified atmosphere. 5×10⁴ to 1.5×10⁵ cells were seeded per well depending on the cell volume to give adequate well-bottom coverage.
The plate was incubated inside a humidity chamber where heat distribution and humidity are optimized to reduce the “edge effect” (bad cell distribution in the well).
9) Human JURL-MK2 chronic leukemia in blast crisis cell line (DSMZ, Germany) was used as a positive control for MiCK assays performed with patient tumor cells.
If a half area 96-well plate is used the total volume per well is 120 μl.
10) RPMI-1640 medium without phenol red was used for all cultures.
11) It was supplemented with 10% fetal bovine serum, 100 units/mL of penicillin, and 100 micrograms/mL of streptomycin.
12) Cell counts and viability were evaluated by trypan blue dye exclusion.
Note: The cell counts and viability checks are done several times during the purification procedure, before adding the cells to the wells of the plate.
13) Each tumor cell preparation, after purification of contaminating and necrotic cells, was analyzed using the diff quick or the Pap stain. This is much improved process allowing one to identify the cell population of interest and verify that there are few remaining contaminating cells.
14) If an adequate number of cells were available, immunocytochemical stains were also performed to better characterize the tumor phenotype.
15) All specimens achieved at least 90% pure tumor cell content by visual estimation by an experienced pathologist and 90% viability by trypan blue exclusion.

### Example 3

### Blood/Bone Marrow Cell Specific Isolation Protocol

Within 24 to 48 hours of collection, the specimen was treated as follows:
Pool the blood into a 50 ml tube.
Take an aliquot for smear.
Do a cell count in acetic acid 2.86% with an hemacytometer.
Take an aliquot for flow cytometry.
Dilute the blood with an equal volume of RPMI.
Do a lymphoprep centrifugation (30 min at 2000 RPM) 4 ml lymphoprep overlaid by up to 8 ml of blood/RPMI mixture.
Collect the mononuclear cell layer, add 10 ml of RPMI 10% FBS and centrifuge at 1500 RPM for 5 min
Resuspend the pellet in 5 ml of RBC lysis solution, incubate 2-3 min and add 5 ml of RPMI 10% FBS, centrifuge for 5 min at 1500.
Resuspend the pellet in RPMI 10% FBS, do a cell count+cytospin.
According to the flow cytometry results, remove unwanted cells with magnetic beads (monocytes=CD14, T lymphocytes=CD2, B lymphocytes=CD19, neutrophils=CD15).
Resuspend the pellet in a small volume of PBS 2% FBS (0.2 to 2 ml).
Wash the beads suspension 3 times with the PBS 2% FBS.
Add the beads to the cell suspension and incubate 30 min at room temperature on the rotator.
Put the tube on the magnet, wait for 1 min
Collect the cell suspension, put in a 15 ml tube with 5 ml of RPMI 10% FBS
Put the tube of the cell suspension on a magnet again to remove remaining beads, collect the cell suspension and put in a new 15 ml tube.
Centrifuge at 1500 RPM for 5 min
Resuspend in RPMI 10% FBS, the volume depends on the pellet size. Do a cell count and determine viability, do a cytospin to determine the cell content.
Take an aliquot for flow cytometry. If the results confirm the purity of the cell population of interest, adjust the cell concentration to approximately 2×10⁶ cells per ml and test the coefficient using the microplate reader. The target value of the coefficient should be between 0.8 and 1.0
Adjust the cell concentration by concentrating or diluting the suspension. Test the coefficient again until a satisfactory value is obtained.
Put the cells in the plate and start the MiCK assay procedure immediately.

### Example 4

### Effusion Specific Isolation Protocol

Within 24 to 48 hours of collection, the specimen was treated as follows:
Transfer the specimen into 50 ml tubes and take also a 10 ml aliquot in a 15 ml tube (centrifuge the aliquot 2000 RPM 5 min, do a cell count and prepare a cytospin to give an idea of the cell content and count of the specimen).
Centrifuge the tubes at 2000 RPM for 15 min
Remove the supernatant but leave ˜5 ml per tube. Combine all the tubes and dilute 1:1 with PBS in as many 50 ml tubes as needed. Centrifuge 10 min at 2000 RPM.
Do RBC lysis for 2-3 min. The volume depends on the pellet size. Add an equal volume of RPMI 10% FBS.
Centrifuge 1500 RPM for 5 min
Resuspend the pellet in RPMI 10% FBS, the volume depends on the pellet size.
Do a cell count and determine viability.
Viability is critical to the entire process. It must be determined if the viability is less than ˜70%. If so, do an optiprep centrifugation.
If the viability meets the acceptable standard, and if the major contaminating cells are macrophages, these cells are removed via adherence.
If there is a high contamination from a major cell type and the total cell count is high (5×10⁷ cells or more), do a first purification step with CD45 beads (1 bead per cell). Then repeat the beads a second time and a third time if necessary.
Do a cell count and determine viability.
Repeat optiprep if necessary as recommended by Pathologist.
Coefficient Adjustment—Adjust the coefficient as for the solid tumor specimen based on recommendation of Pathologist.
When the optimal cell concentration is reached, put the cells in the plate and incubate overnight in the incubating chamber of the incubator (37° C.).

### Example 5

### Modified MiCK Assay for Evaluating Apoptosis Mediated by Anti-Cancer Drug Candidates

The MiCK assay procedure was adapted from the method described in U.S. Pat. No. 6,077,684 and U.S. Pat. No. 6,258,553, both patents incorporated herein by reference in their entirety. Also, the MiCK assays described in: Kravtsov V. et al. Use of the Microculture Kinetic Assay of apoptosis to determine chemosensitivities of leukemias. Blood 1998; 92: 968-980, is incorporated herein by reference in its entirety for all purposes. The specific MiCK assay protocols utilized are described in examples 1-4.

After overnight incubation, chemotherapy drugs were added to the wells of the 96-well plate in 5 microliter aliquots or to the wells of a 384-well plate in 2.5 microliter aliquots using an automated pipettor. The number of drugs or drug combinations and the number of concentrations tested depended on the number of viable malignant cells that were isolated from the tumor specimen. The drug concentrations, determined by molarity, were those indicated by the manufacturer as the desired blood level concentration plus or minus one serial dilution if enough cells were available.

Following drug addition, the plate was incubated for 30 min at 37° C. into a 5% carbon dioxide humidified atmosphere incubator. Each well was then overlayed with sterile mineral oil, and the plate was placed into the incubator chamber of a microplate spectrophotometric reader. The optical density at 600 nanometers was read and recorded every 5 minutes over a period of 48 hours. Optical density increases, which correlate with apoptosis, were converted to kinetic units (KU) of apoptosis by a proprietary software ProApo with a formula described in the previous Kravtsov reference incorporated by reference (i.e. Kravtsov V. et al. Use of the Microculture Kinetic Assay of apoptosis to determine chemosensitivitis of leukemias. Blood 1998; 92: 968-980) and were correlated with patient outcomes. Active apoptosis was indicated as >1.0 KU. A drug producing ≦1 KU was described as inactive, or that the tumor was resistant to that drug based on previous laboratory correlations of KU with other markers of drug-induced cytotoxicity (growth in culture, thymidine uptake).

Treatment of Patients with Data Obtained from MiCK Assay of Present Disclosure

The aforementioned study and associated MiCK protocol was a prospective multi-institutional non-blinded trial. MiCK assay results obtained before any therapy was initiated were always transmitted to physicians. Physicians treated patients with the physicians' own choice of drugs as they deemed clinically indicated and were free to use or not use any of the data from the MiCK assay. Tumor responses were measured by RECIST or other clinical criteria. Patients were evaluated for time to recurrence after assay and survival after assay.

There were no rules or directions about how to use the MiCK assay results. The study evaluated whether the oncologist used the results of the assay, whether other data was also used (e.g., estrogen receptor analysis or Her2 test results, or addition of other drugs) or whether the assay results were not used. Because instructions or rules about using the assay were not given, it was felt that this was a more valid test of how the assay would be used in the “real world” where oncologist have complete discretion in treatment planning

### Statistical Evaluation

One of the goals of the study was to identify how frequently physicians used the MiCK assay results to help determine patient treatment, and to correlate use of the MiCK assay with response rate, relapse-free interval, and overall survival. Physicians completed questionnaires in which they described what the intended treatment was before the assay data was returned, what treatment was used after the assay was reported, and whether the assay was used in formulating the final treatment given to the patient. Data were imported into SAS software for analysis. If a sample had multiple doses of the same drug, then the concentrations with the highest KU value was assigned to the drug. Nonparametric Kaplan-Meier product limit methods were used for survival analysis and the analysis of relapse-free interval. In this analysis the log rank test was used to compare survival curves and the Wilcoxon test for comparing medians. Response rates were compared using contingency tables and Fisher's exact test.

### Investigational Review Board Approval

Investigators performed this trial after IRB approval was obtained from and monitored by the Western IRB in Seattle, Wash. Each patient had given voluntary informed consent in writing prior to submission of tumor specimen for MiCK analysis. The clinical trial was registered at clinicaltrials.gov NCT00901264.

### Results

The patient characteristics are described in Table 3. Mean age was over 65, and 29 patients were female. A variety of tumors were studied, including breast (16), non-small cell lung cancer (6), non-Hodgkin's lymphoma (4) and others. Physicians most commonly entered patients who were being considered for palliative chemotherapy. Only 4 patients were entered who were being considered for adjuvant chemotherapy. The median line of therapy planned to be used for palliative care after the MiCK assay was 2^{nd} line, with a range of first line treatment up to 8^{th} line treatment. The median time of follow up for patients was 4.5 months (4.0 months in patients whose physicians did not use the MiCK assay, versus 5.6 months in patients whose physicians used the MiCK assay to plan the treatments).

MiCK assay results were frequently used by physicians (Table 4). 64% of patients received chemotherapy based at least in part on the MiCK assay. 18 (41%) used only the MiCK assay. In 10 patients (23%), physicians used MiCK results but also combined that information with other drugs not tested in the assay, or modified the assay results based on individual patient characteristics such as organ function and based on tumor biological characteristics. The biological characteristics of these varied tumors were considered by the oncologists in developing the final treatment plans. For example, in breast cancer, hormone-receptor positive patients received hormonal agents in addition to chemotherapy, and trastuzumab in addition to chemotherapy in Her2 positive patients. Patients with non-small cell lung cancer who were egfr-mutation positive received erlotnib prior to consideration for performing the drug-induced apoptosis assay. CD20 positive non-Hodgkin's lymphoma patients received rituximab in addition to chemotherapy. In 22 patients (50%), a change in chemotherapy resulted based on using the MiCK assay results.

Even though patients had signed consent to obtain the assay, in 16 instances the physician did not use the assay to determine patient treatment. In 1 instance the patient entered a clinical trial. After being advised of the assay results and proposed treatment based on the assay, 7 patients preferred to be treated with another therapy (usually due to toxicity of the therapy identified as best in the MiCK assay). In the other 8 patients, the physician preferred to use another treatment based on literature or physician's personal experience.

In breast cancer, the largest subset of patients that were treated, 9/16 [56%] of patients were treated based upon the MiCK assay. In 3/9, the MiCK assay was used with other non-tested drugs, in 3/9 MiCK results were combined with targeted biotherapies, in 2/9, MiCK results were combined with hormonal therapy, and in 1/9 only the drugs active in the MiCK assay were used.

Effect on Choices of Chemotherapy, Generic Vs. Proprietary

In 16 patients (36%), oncologists changed from an intended use of proprietary chemotherapy before knowledge of the MiCK assay to actual use of generic drugs after assay results were reviewed. In 3 (7%) of patients, physicians changed from intended use of generic drugs to actual use of proprietary drugs. In 9 patients (20%), physicians used single drug therapy after the MiCK assay, compared to an intended use of combination therapy prior to knowing MiCK assay results. In 4 patients (9%), oncologists used combination therapy after MiCK assay results, compared to an intended use of single drugs prior to knowledge of the MiCK assay results.

When physicians used the MiCK assay, they used a chemotherapy that produced the highest KU value in 16 patients. Physicians used a treatment with a higher degree of apoptosis (greater than 2 KU) in 23 patients.

### Effect on Patient Outcomes

In patients receiving palliative chemotherapy, complete plus partial response rates were compared to the use or non-use of the MiCK assay (Table 5). If physicians used the results of the MiCK assay, complete plus partial response rate was 44%. This compared to only 6.7% CR plus PR rate if physicians did not use the MiCK assay (p<0.02).

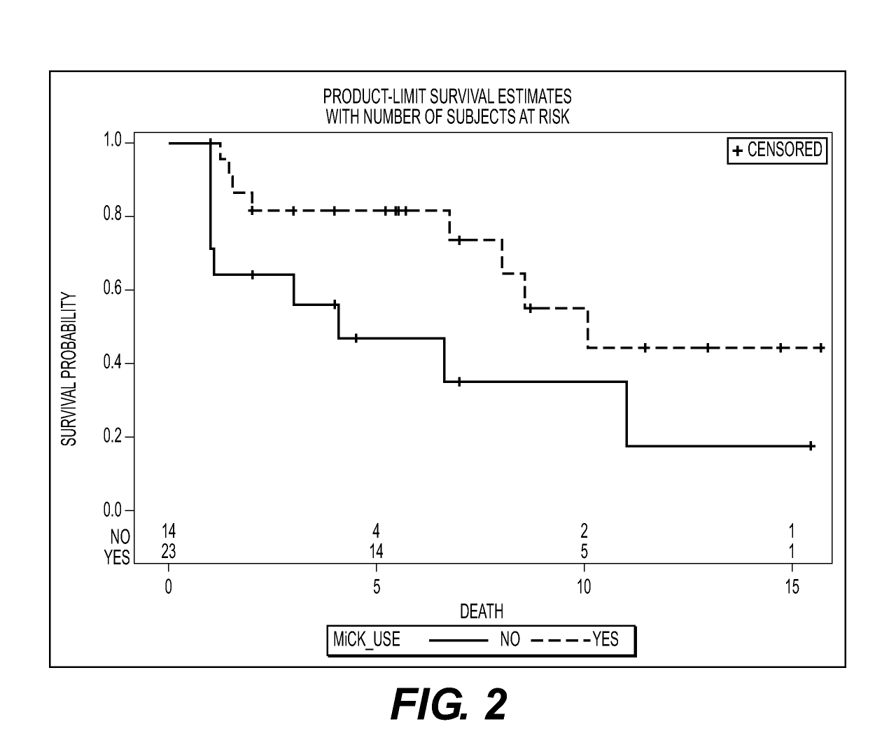

Overall survival was compared to use or non-use of the MiCK assay results (FIG. 2). If physicians used the MiCK assay for determination of patient therapy, median overall survival was 10.1 months compared to only 4.1 months if physicians did not use MiCK assay results (p=0.02).

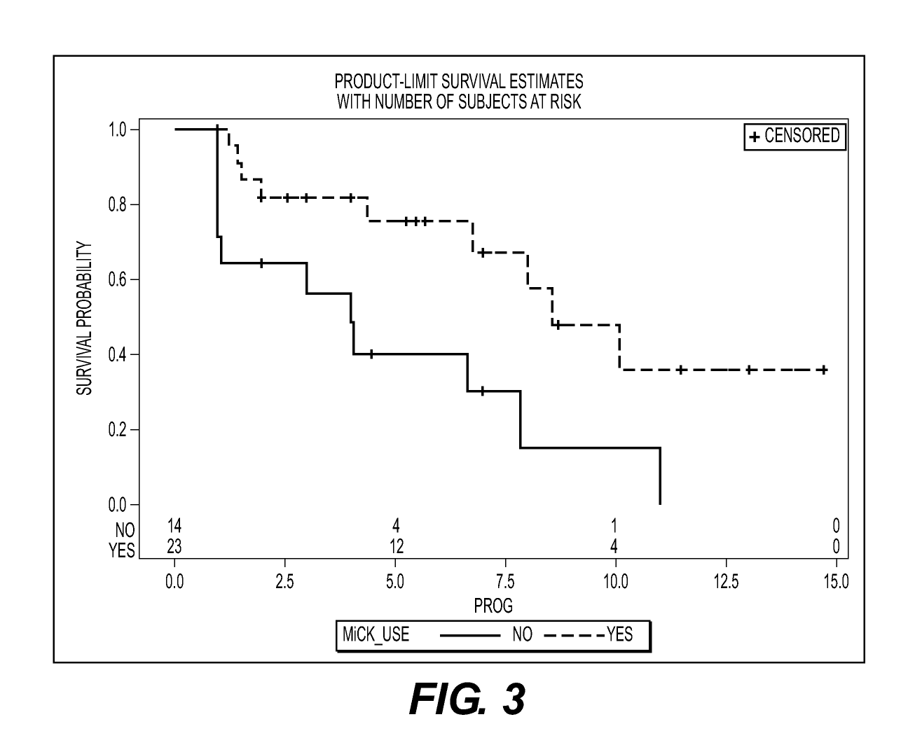

The relapse-free interval in patients whose physicians used the MiCK assay to determine therapy was compared to those patients whose physicians did not use the MiCK assay results (FIG. 3). The median relapse-free interval was 8.6 months in patients whose physicians used the MiCK assay, compared to 4.0 months in patients whose physicians did not use the MiCK assay (p<0.01).

In order to rule out the possibility that the addition of other drugs to the chemotherapy selected based on the MiCK assay was responsible for the advantages observed when oncologists used the MiCK assay, we compared the results of patients whose oncologists used only the MiCK assay with the results of patients whose oncologists did not use the MiCK assay. Complete and partial response rates were higher in patients treated based only on the MiCK assay (43.8%) compared to patients treated without the use of the MiCK assay (6.7%, p=0.04). Overall survival was longer in patients treated based only on the MiCK assay (median 10.1 months) compared to patients treated without the use of the MiCK assay (median 4.1 months, p=0.02). The relapse-free interval was longer in patients treated based only on the MiCK assay (median 8.0 months) compared to patients treated without the use of the MiCK assay (median 4.0 months, p=0.03). Thus, we conclude that the use of the MiCK assay (and not the addition of other drugs) was associated with the improved outcomes observed.

### Discussion

This utility study was non-blinded, so that the oncologist received, within 72 hours of biopsy, the drug-induced apoptosis results and a laboratory interpretation of which therapies were best in vitro, and the actual KU of apoptosis for each single drug or combination tested.

Results demonstrate that the MiCK assay was frequently used by physicians to determine patient treatments. The 64% rate of use of this predictive bioassay by oncologists, to design the chemotherapy treatment plan, was considered to be evidence of clinical utility (physicians will use the results in patient care).

The results in this study indicate that not only are oncologists willing to use the results of the assay, but when they do, outcomes are likely to be superior to results when physicians do not use the assay. The magnitude of the improvement in these patients was large enough to be statistically significant.

This finding of improved outcomes may also reduce costs of care by avoiding use of less effective treatments. The observation that physicians often used less costly generic drugs may be important to oncologists by suggesting when generic drugs might be at least as useful as proprietary drugs.

Thus, when physicians are informed of the MiCK assay results, they frequently use the results to plan patient treatments. When physicians use the results, patient outcomes appear to be better.

### Example 6

### Patterns of In Vitro Chemotherapy (CT)-Induced Apoptosis (APOP) in Recurrent/Metastatic Breast Carcinoma (CA): Comparisons of Generic Multi-Source Drugs (Generics) with Proprietary Single-Source Drugs (Proprietaries)

### Experimental Background

Therapy of metastatic breast cancer involves choices between Generics and Proprietaries, and between combination chemotherapies (Combos) and single-agent chemotherapies. This experiment determined the relative in vitro chemotherapy induced apoptosis of Generics versus Proprietaries, and Combos versus single agents.

### Methods

Purified breast cancer cells from 67 patient (Pt) biopsies were placed in short-term culture with chemotherapy using the microculture kinetic (MiCK) assay described in examples 1-4. Apoptosis was analyzed every five min over 48 hrs. Apoptosis was defined in kinetic units (KU) of apoptosis. Significant Apoptosis was >1.0 KU. Significant difference between individual assays was >0.57 KU based on replicate analyses.

Drugs were classified as generic (g) or proprietary (p) based on the following scheme:

Generic=5-fluorouracil, carboblatin, cisplatin, cytoxan, doxorubicin, etoposide, epirubicin, ifosfamide, methotrexate, mitoxantrone, taxol, taxotere, vincristine, vinorelbine, vinblastine.

Proprietary=abraxane, doxil, eribulin, gemzar, ixabepilone, oxaliplatin, xeloda

### Results

43 patients (pts) were evaluable for comparison of Generics versus Proprietaries. Generics produced APOP>Proprietaries in 36/43 Pts (84%) and = to Proprietaries in 6 Pts (14%). Proprietaries produced APOP>Generics in 1 Pt (2%). These results are illustrated in Tables 6 and 16. Also, Table 7 further illustrates the patient characteristics of the breast cancer specimens.

In-class comparisons indicated epirubicin had mean APOP>doxorubicin (P=0.01), cisplatin had APOP>carboplatin (P<0.01); vinorelbine had APOP>vincristine (P=0.02); docetaxel had APOP>nab-paclitaxel (P=0.01); whereas docetaxel and paclitaxel APOP were not different (P=0.85). These and other detailed comparisons may be found in Tables 8-33.

However, in individual Pts, docetaxel had APOP>paclitaxel in 37% of Pts, whereas paclitaxel was better than docetaxel in 31%. For Combos, cyclophosphamide+doxorubicin produced APOP>single agents in 25%, while single agents had APOP= or >cyclophosphamide plus doxorubicin in 67%. Cyclophosphamide plus docetaxel had APOP>single agents in 33%, but single agents had APOP= or >cyclophosphamide plus docetaxel in 66%. These and other detailed comparisons may be found in Tables 8-33.

### Conclusions

Generics APOP is often equal to or better than Proprietaries APOP. In individual patients single agents frequently produced higher APOP than Combos. The currently disclosed MiCK APOP assay can identify individual Pts with metastatic breast CA for whom Generics or single agents produce higher APOP than Proprietaries or Combos. These differences could result in significant savings in health care costs.

### Example 7

### Are Generic Multi-Source (Generic) Chemotherapy (CT) Drugs as Effective as Proprietary Single-Source (Proprietary) Drugs? Evidence from In Vitro CT-Induced Apoptosis (APOP) in Non-Small Cell Lung Cancer (NSCLC), Colorectal Cancer (Colon CA) Compared to Recurrent/Metastatic Breast Carcinoma (Breast CA)

### Experimental Background

We have demonstrated that cancer cells from patients (Pts) with recurrent or metastatic Breast cancer frequently show as much or better apoptosis with Generics compared to Proprietaries (Example 6 discussed above). We have compared these observations to in vitro apoptosis in patients with NSCLC and Colon cancer.

### Methods

Purified tumor cells from patient biopsies were placed into short term culture using the microculture kinetic (MiCK) assay described in examples 1-4. Apoptosis was analyzed every five minutes over 48 hours. apoptosis was defined in kinetic units (KU) of apoptosis. Significant apoptosis was >1.0 KU, significant differences between individual assays were defined as >0.57 KU based on replicate analyses. Results from Breast CA, Colon CA and NSCLC were compared.

Drugs were classified as generic (g) or proprietary (p) based on the following scheme:

Generic=Cytoxan, 5-fluorouracil, cytarabine, carboplatin, carboplatin/Taxol, carboplatin/Taxotere, cisplatin, cisplatin/Taxol, cisplatin/Taxotere, epirubicin/etoposide, etoposide, idarubicin, ifosfamide, irinotecan, melphalan, methotrexate, mitomycin, mitoxantrone, topotecan, vinblastine, vincristine, vinorelbine.

Proprietary=5-fluorouracil/irinotecan/oxaliplatin, 5-fluorouracil/oxaliplatin, Alimta, Alimta/Taxol, Alimta/carboplatin, Alimta/cisplatin, cisplatin/Gemzar, irinotecan/Xeloda, Alimta/Gemzar, Gleevec, oxaliplatin/Xeloda, sorafenib, sunitinib, Tarceva, Xeloda, Abraxane, Gemzar, oxaliplatin.

### Results

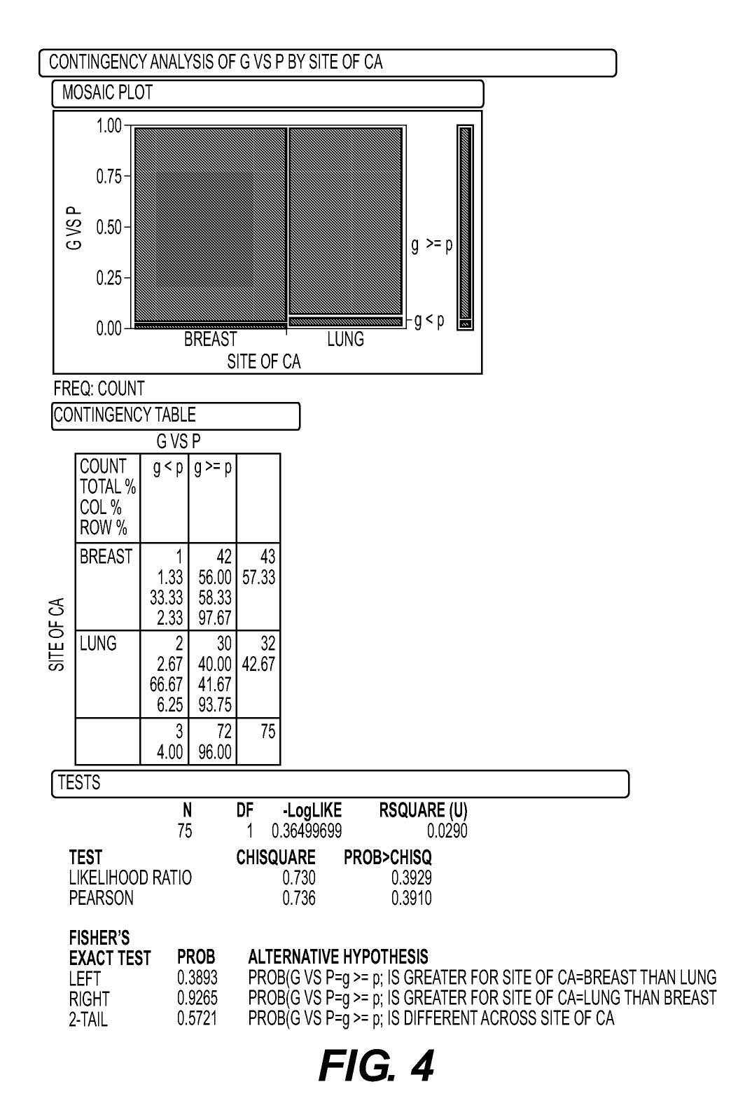

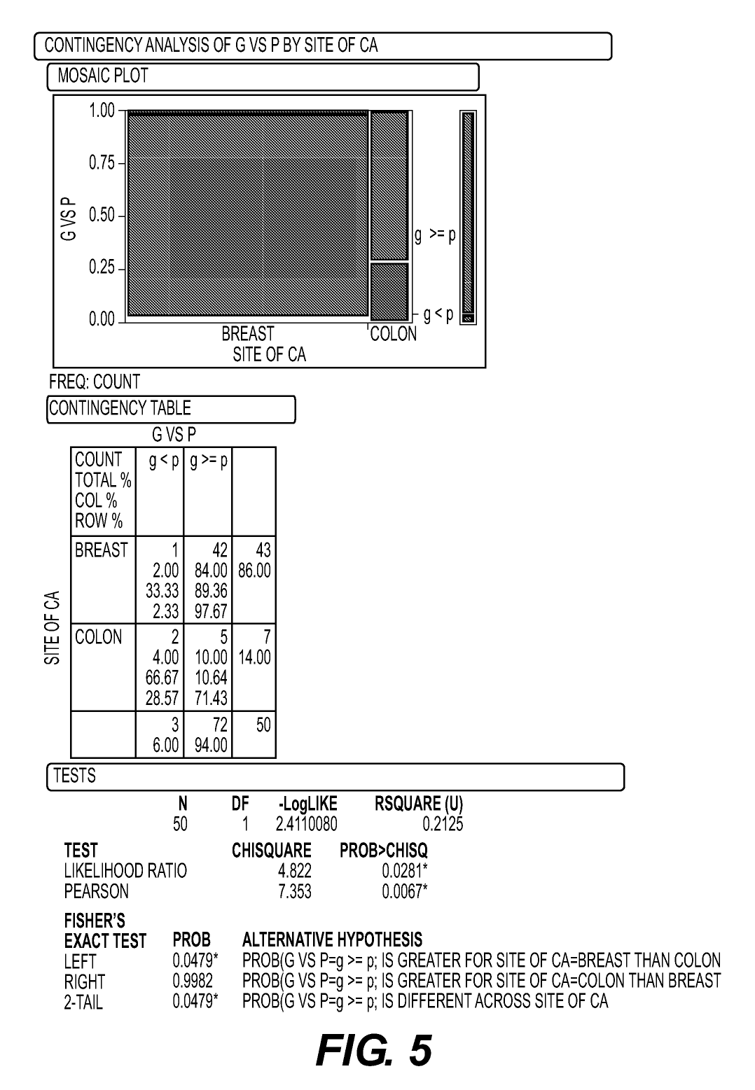

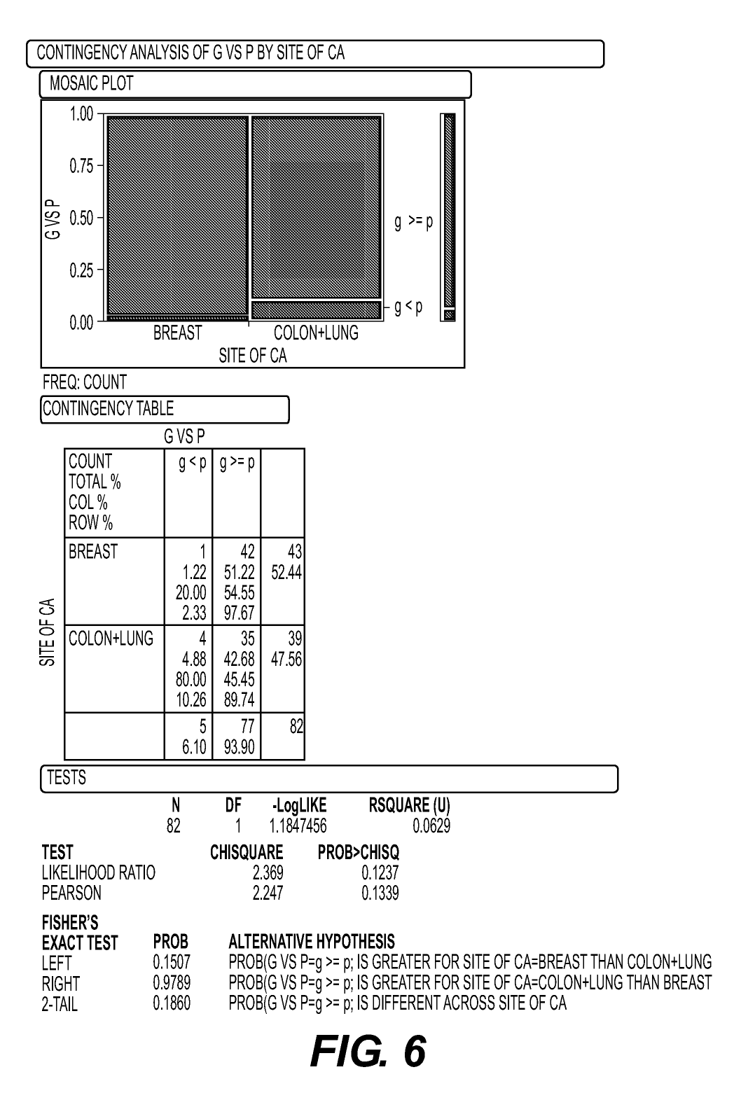

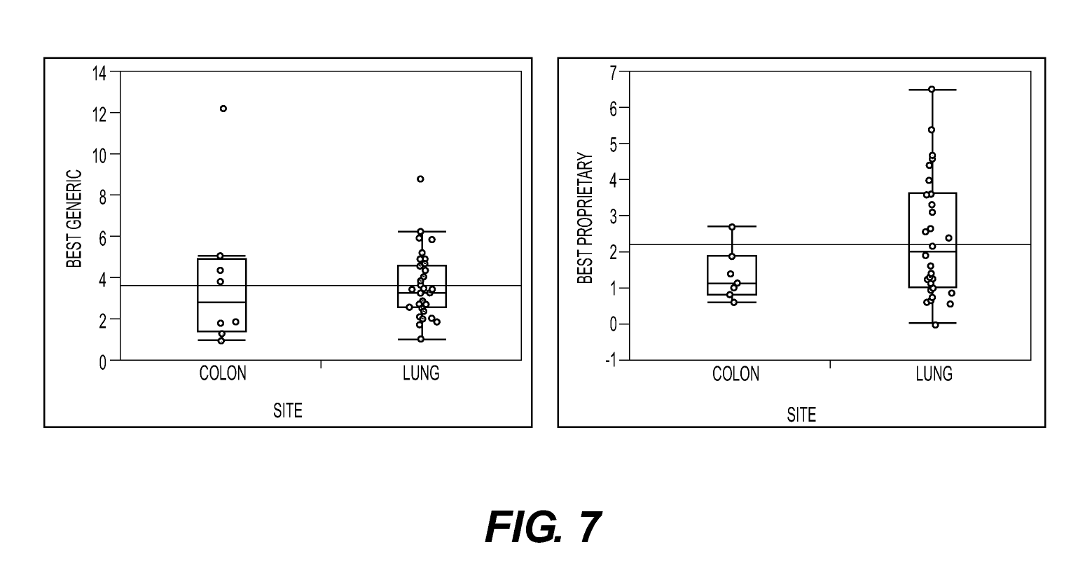

41 patients (pts) with NSCLC, 8 Pts with Colon CA and 67 Pts with Breast CA had successful cultures. Generics produced APOP greater than Proprietaries in 25/32 Pts with NSCLC (78%), 4/7 Pts with Colon CA (57%) and 36/43 Pts (84%) with Breast CA. Generics produced APOP=Proprietaries in 5 Pts with NSCLC (16%), 1 Pt with Colon CA (14%) and 6 Pts (14%) with Breast CA. Proprietaries produced APOP greater than Generics in 2 Pts with NSCLC (6%), 2 Pts with Colon CA (29%) and 1 Pt (2%) with Breast CA. There were 0 Pts with NSCLC, Colon CA or Breast CA in whom no drug produced significant APOP (KU less than 1.0). Proprietaries produced more APOP in Colon CA than in Breast CA (p<0.05). These results can be found in: Table 6 (all diseases specimens); Table 16 (Breast cancer specimens); Table 34 (Lung cancer specimens); and Table 35 (Colon cancer specimens). A comparison of the statistical significance between the tested tissue specimen types, in relation to whether generics or proprietary drugs are more effective, can be found in FIGS. 4-7.

### Conclusions

Generic drugs can produce APOP in vitro equal to or better than Proprietary drugs in most Pts with NSCLC, Colon CA, and Breast CA. The frequency of Generic drugs being at least as active as Proprietary drugs varies by disease, and was higher in Breast CA compared to Colon CA. However, the MiCK APOP assay can identify which individual Pts might require use of Proprietary drugs. These conclusions justify prospective clinical trials to confirm these in vitro results. Increased use of Generic drugs based on the APOP assay may help to control healthcare costs.

### Example 8

### Cost Savings by Use of a Chemotherapy-Induced Apoptosis Assay in Breast, Colon and Non-Small Cell Lung Cancers

### Experimental Background

Chemotherapy costs in the United States have become dramatically high. We have demonstrated in the preceding examples 1-7 that an improved chemotherapy-induced apoptosis assay (the microculture-kinetic, or MiCK assay) has been developed. Use of the assay to plan chemotherapy treatment was shown to be associated with improvement in clinical outcomes: improved response rate, longer time to relapse, and longer survival (Example 5). The previously presented experiments also indicated that in the assay, the drug-induced apoptosis from generic multi-source drugs was frequently greater than or equivalent to the apoptosis from proprietary single-source drugs (Examples 5-7). Therefore, this experiment was performed to estimate the possible cost savings by using the MiCK assay to substitute generic multi-source drugs for proprietary single-source drugs in treating patients with breast, colon, and non-small cell lung cancers. We use the generic term, monetary consequences, to denote the monetary differences which would result from utilizing one drug candidate versus another. These monetary consequences can be beneficial to a patient or healthcare system if, for example, the chosen drug (often a generic) is relatively cheaper than a compared proprietary counterpart. In a scenario in which the chosen generic drug is cheaper than its proprietary counterpart, one would term the monetary consequence (for example the difference in cost between using the generic and proprietary), as a cost savings. However, the monetary consequences do not have to result in a cost savings, because the drug with the higher KU value could be the drug candidate which costs relatively more money. In that situation, the monetary consequence of choosing the drug candidate to use for a patient based upon the MiCK assay would result in a relative loss of money, as a more expensive drug would be chosen. The generic monetary consequences term may also be further described by utilizing the Mean Drug Savings, Assay Adjusted Mean Drug Savings, and Net Mean Drug Savings statistics elaborated below.

### Methods

Purified tumor cells from Pt biopsies were placed into short term culture using the microculture kinetic (MiCK) assay described in examples 1-4. Namely, Sterile tumor specimens with at least 0.5 cm³ of viable tumor tissue, 5 core needle biopsies, or 1000 ml of malignant effusions were obtained. Within 24 to 48 hours of collection, the specimen was minced, digested with 0.25% trypsin and 0.08% DNase for 1-2 hours at 37 C.°, and then filtered through a 100 micrometer cell strainer. When necessary, non-viable cells were removed by density gradient centrifugation. The cell suspension was then incubated for 30 min at 37° C. in a tissue culture flask to remove macrophages by adherence. For epithelial tumors lymphocytes were removed by 30 minute incubation with CD2 antibody conjugated magnetic beads for T lymphocytes and CD19 antibody conjugated magnetic beads for B lymphocytes. Remaining macrophages were removed, if necessary, using CD14 antibody conjugated magnetic beads. The final cell suspension was plated into a 96-well or 384-well half-area plate, 120 microliter aliquot per well. The plate was incubated overnight at 37° C. with 5% carbon dioxide humidified atmosphere. 5×10⁴ to 1.5×10⁵ cells were seeded per well depending on the cell volume to give complete well-bottom coverage. Human JURL-MK2 chronic leukemia in blast crisis cell line (DSMZ, Germany) was used as a positive control for MiCK assays performed with patient tumor cells. RPMI-1640 medium without phenol red was used for all cultures. It was supplemented with 10% fetal bovine serum, 100 units/ml of penicillin, and 100 micrograms/ml of streptomycin. Cell counts and viability were evaluated by trypan blue dye exclusion. After purification of contaminating and necrotic cells, each tumor cell preparation was analyzed by a pathologist using hematoxylin/eosin stained cytospin preparations to confirm the presence of malignancy cytologically. If an adequate number of cells were available, immunocytochemichal stains were also performed to better characterize the tumor phenotype. To be evaluable, tumor specimens contained at least 90% tumor cell content by pathology evaluation and 90% viability by trypan blue exclusion.

After overnight incubation, chemotherapy drugs were added to the wells of the 96-wellplate in 5 microliter aliquots. The number of drugs or drug combinations and the number of concentrations tested depended on the number of viable malignant cells that were isolated from the tumor specimen. The drug concentrations, determined by molarity, were those indicated by the manufacturer as the desired blood level concentration plus or minus one serial dilution if enough cells were available. Following drug addition, the plate was incubated for 30 min at 37° C. into a 5% carbon dioxide humidified atmosphere incubator. Each well was then overlaid with sterile mineral oil, and the plate was placed into the incubator chamber of a microplate spectrophotometric reader (BioTek instruments). The optical density at 600 nanometers was read and recorded every 5 minutes over a period of 48 hours. Optical density increases, which correlate with apoptosis, were converted to kinetic units (KU) of apoptosis by a proprietary software ProApo with a formula described above. Active apoptosis was indicated as >1.0 KU. A drug producing ≦1 KU was described as inactive, or that the tumor was resistant to that drug based on previous laboratory correlations of KU with other markers of drug-induced cytotoxicity (growth in culture, thymidine uptake).

Results of all assays from patient with breast carcinoma with recurrent disease, colon carcinoma, or non-small cell lung carcinoma that had been completed by the study cut-off date were analyzed. Studies were evaluable only if both proprietary single-source drugs and generic multi-source drugs were both tested in the assay. Superiority of a drug was defined as apoptosis 0.57 KU or more above the comparative drug. Equivalence was defined as apoptosis for one drug within 0.57 KU of a second drug. Inferiority was defined as apoptosis for one drug 0.57 units or more below the second drug.

Costs of chemotherapy were evaluated using Medicare payments for 6 cycles of therapy (based on the payment schedule for the fourth quarter 2011). A chemotherapy cycle consisted of 3 or 4 weeks of therapy (depending on the drug or combination). Patients were assumed to be 1.8 m² in surface area, because this is the average size of a human being. This measurement was used to calculate the dosage of the drug.

Proprietary single source drugs were nab-paclitaxel, gemcitabine, oxaliplatin, capcitabine, ixabepilone, erubilin, liposomal doxorubicin, and pemetrexed.

Generic multisource drugs were cyclophosphamide, doxorubicin, epirubicin, paclitaxel, docetaxel, cisplatin, carboplatin, irinotecan, topotecan, vinorelbine, and vinblastine.

Proprietary drugs or combinations for breast cancer were nab-paclitaxel, capcitabine, and gemcitabine; for colon cancer was 5-fluorouracil plus leucovorin plus oxaliplatin; and for non-small cell lung cancer were pemetrexed plus cisplatin and gemcitabine plus cisplatin.

Generic drugs or combinations for breast cancer were vinorelbine, docetaxel plus cyclophosphamide, and epirubicin plus cyclophosphamide; for colon cancer was 5-fluorouracil plus leucovorin plus irinotecan; and for non-small cell lung cancer were carboplatin plus paclitaxel, vinorelbine, or docetaxel.

The medicare reimbursement for 6 cycles of each drug or combination was calculated and the average of proprietary drugs and average for generic drugs for each cancer were then compared.

The mean drug savings was defined as the difference between the mean proprietary drug cost minus the mean generic drug cost. The assay-adjusted mean drug savings was defined as the drug savings multiplied by the frequency of generic drug superiority or equivalence to proprietary drugs (as determined by the MiCK assays). The net mean drug savings was defined as the assay-adjusted mean drug savings minus $5000, the estimated cost of the MiCK assay. The percent cost savings was defined as net drug savings divided by mean proprietary drug cost. The following formulas illustrate these relationships:
Mean Drug Savings=mean proprietary drug cost−mean generic drug cost
Assay Adjusted Mean Drug Savings=(mean proprietary drug cost−mean generic drug cost)×frequency of generic drug superiority or equivalence to proprietary drugs
Net Mean Drug Savings=(mean proprietary drug cost−mean generic drug cost)×frequency of generic drug superiority or equivalence to proprietary drugs−cost of MiCK Assay

### Statistical Analyses

A determination was made as to the three most widely used treatment programs for each cancer. Then, the standard average dosage for each treatment was determined, as well as the medicare allowable cost for each cancer for an individual patient. Then, MiCK assays were run and the results allowed an ascertainment of the best treatment plan based on the various cancer types. These MiCK assay deduced best treatment plans were then compared to the usual treatment costs. Following the comparison, the results and selected best treatment plans based upon the MiCK assay results were reviewed by a nationally recognized cancer cost consultant.

### Results

There were 7 patients with colon carcinoma, 32 patients with non-small cell lung carcinoma and 43 patients with breast carcinoma who were evaluable (Table 6 and as presented in Example 7). The table indicates that generic multi-source drugs were equal to or greater than proprietary single-source drugs in 71% in colon cancer, 98% in breast cancer and 94% in non-small cell lung cancer. Proprietary drugs produced more drug-induced apoptosis in 29% of patients with colon cancer, 2% in patients with breast cancer and 6% with patients with non-small cell lung cancer.

The cost of care for drugs was then modeled as described in the methods. The results indicated that the differences in costs for six months of care for drugs alone (excluding chemotherapy administration, supportive care drugs, tumor testing, hospitalization, or emergency care) were as listed in Table 36 and Table 37.

In all 3 cancers there were substantial savings by substituting generic drugs for proprietary drugs.

The assay-adjusted mean drug savings remained high for each of the cancers (Table 36). The estimated net savings per patient varied from $8,321 to $20,338. Percent cost savings varied from 42.8% to 54%. Based on the methods of the present invention, breast cancer treatments would witness a 43% savings; colon cancer treatments would witness a 54% savings; and non-small cell lung cancer treatments would witness a 47% savings.

### Discussion

This study indicates that use of the drug-induced apoptosis assay, of an embodiment of the present invention, could result in substantial cost savings (Table 36). This assumes that all physicians in the absence of the assay would use proprietary drugs or combinations, and that when a physician was aware of the results of the assay, the physician would follow the guidance of the assay and use generic drugs or combinations if they were better than or equal to proprietary drugs and combinations, and use proprietary drugs or combinations if they were superior in the assay.

This study assumes that all physicians would use drugs that were best in the drug-induced apoptosis assay. In a previous example (Example 5), it was found that the physicians used the best results from the drug-induced apoptosis assay 64% of the time. Therefore, it is possible that the net cost savings (estimated in the Table 36) might be reduced by as much as 36%. However, as the prior study in example 5 progressed, increasing numbers of physicians followed the guidance of the assay, indicating that the 64% rate of usage of results from the drug-induced apoptosis assay is probably a minimal estimate.

The potential cost savings must also be acknowledged to be only for the chemotherapeutic drugs tested in the assay. As more proprietary drugs become available in certain diseases (e.g. breast cancer), it is possible that an increasing percentage of patients may be more responsive to proprietary drugs, and net cost savings would therefore be less. It is also possible that some proprietary drugs would become generic (e.g. colon cancer), thus, possibly reducing differential cost and reducing the potential cost savings impact of the use of the assay.

Nevertheless, this study suggests that more widespread use of the drug-induced apoptosis assay of an embodiment of the present invention is highly likely to result in substantial cost savings to patients and to health plans if implemented widely in the oncology community. More importantly, not only would costs be less, but as indicated in Example 5, patient outcomes were better when physicians used an embodiment of the currently disclosed MiCK assay to plan patient therapy. Use of a MiCK assay, according to an embodiment of the present invention, was associated with statistically significantly higher complete and partial response rates, longer time to relapse, and longer survival (Example 5).

Thus, utilization of the currently disclosed MiCK drug-induced apoptosis assay may enable the identification of the dominant therapy for each patient with breast, colon, and lung cancer. Therapy chosen with the utilization of the currently disclosed assay has a better outcome and also lower cost. The presently described MiCK assay will be an important tool in health care reform and personalized medicine.

### Example 9

### Photomicroscopy Experiment

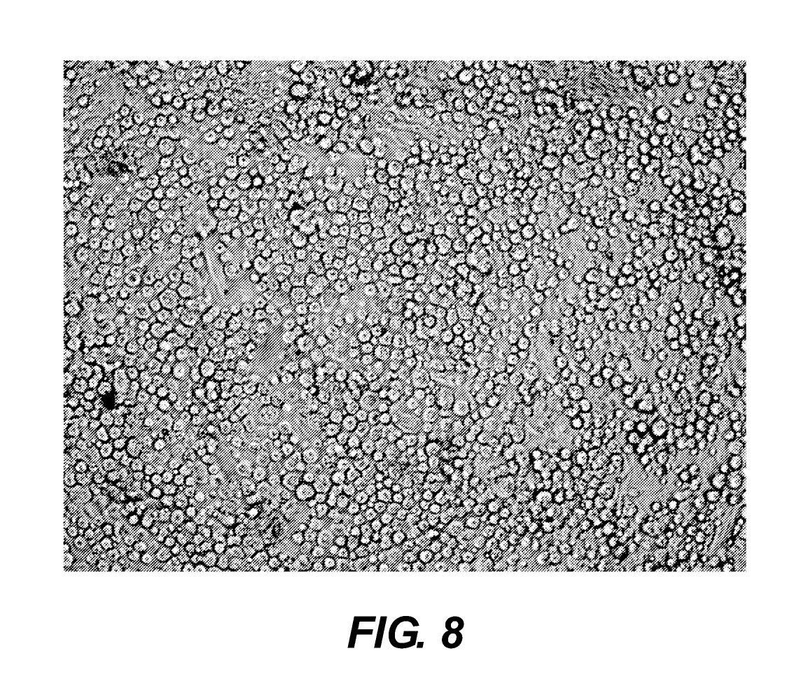

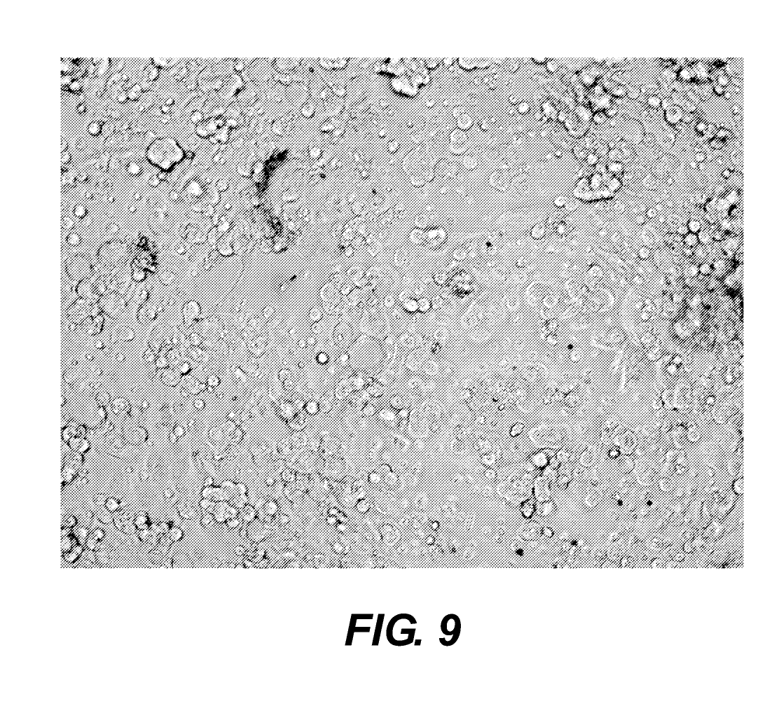

An experiment was conducted to validate the use of photomicroscopy in the methods as claimed. The photomicrographs (FIGS. 8 and 9) illustrate the cell distribution and viability of cells before overnight incubation and after overnight incubation, respectively. Therefore, photomicrographs may be used to assess cell viability and can be considered the last step in the cell isolation/purification process or could be considered the beginning of the MiCK assay.

FIG. 8 is a photomicrograph of cells in one well of a plate before overnight incubation. FIG. 9 is a photomicrograph of the same well after an overnight incubation of 15 hours. The cells in FIG. 9 appear to be more oval and slightly flatter, because they are now adhering to the bottom of the well. FIG. 9 represents the condition of cells in a well, at a point in the method, at which anti-cancer drug candidates are now ready to be added to the well.

### Example 10

### Patient Specific Cancer Cell Testing

An experiment was conducted to ascertain which potential anti-cancer drug candidate would be most effective for a particular patient. The experiment thus validates the disclosed methodology and assays as an effective tool to create individualized cancer treatment protocols.

The experiments were conducted on neoplastic cells collected from spleen and abdominal tumor biopsy specimens from a 55 year old female. The tumor specimens were of an unknown primary. The experiment consisted of using a MiCK assay, according to the present disclosure, to test the effectiveness of 37 potential anti-cancer drugs, combinations of these drugs, and various concentrations of these drugs.

Based on the results, cisplatin is the single drug with the most efficacy for this patient. Cisplatin had a KU value greater than 10KU's (Table 38). However, any of the platinum based drugs utilized as single agents would be highly effective. Sunitinib or Cytoxan, as nonplatinum based drugs, also gave highly effective results and would be good alternatives if the patient could not tolerate platinum.

Apoptotic readings greater than 5.0KU in the MiCK assay are considered to be highly sensitive and are associated with a good clinical response. All reagents and combinations of reagents were control tested against a viable control cell line and found to induce appropriate levels of apoptosis. It should be noted that the alkylating agents cyclophosphamide and ifosfamide require hepatic metabolic transformation to their active metabolite, 4HC and 4HI respectively, and therefore cannot be tested directly in vitro. For the MiCK assay their active metabolites, 4HC and 4HI respectively were used.

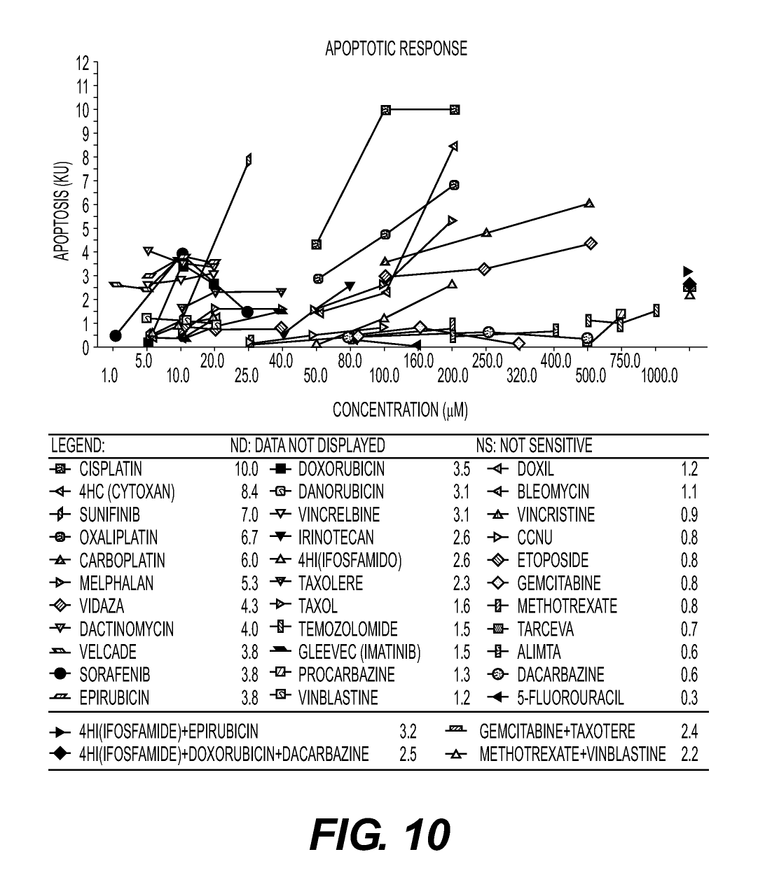

The experiment also tested various concentrations of the 37 anti-cancer drug candidates and this data may be found in FIG. 10. It can be observed that some of the tested anti-cancer drugs had a heterogeneous response on apoptosis depending upon concentration, whereas other drug candidates showed no response with varying concentration.

TABLE 1
Enzyme Utilization Dependent Upon Tumor Type of Specimen

| | | Other enzyme |
|---|---|---|
| | First choice enzyme + | possibility + |
| Tumor type | DNase 0.008% | DNase 0.008% |
| Bladder | Collagenase IV 300 U/ml | |
| Breast | Collagenase IV 300 U/ml | Collagenase III 200 U/ml |
| Cervix | Trypsin 0.25% | |
| Colon | Collagenase I 300 U/ml + | Trypsin 0.25% |
| | Dispase 1 U/ml | |
| Endometrial | Trypsin 0.25% | — |
| Kidney | Collagenase IV 300 U/ml | — |
| Gastric | Trypsin 0.25% | — |
| Leiomyosarcoma | Trypsin 0.25% | Collagenase IV 300 U/ml |
| Liver | Collagenase IV 300 U/ml | |
| Lung | Collagenase IV 300 U/ml | — |
| Melanoma | Collagenase IV 300 U/ml | |
| Ovarian | Trypsin 0.25% | — |
| Pancreas | Collagenase IV 300 U/ml + | — |
| | Hyaluronidase 0.1 U/ml | |
| Prostate | Collagenase I 300 U/ml | — |
| Soft tissue | Trypsin 0.25% | — |
| sarcoma | | |
| Thymus | Collagenase I 300 U/ml | — |

TABLE 2
Final Cell Suspension Plating Protocol

| | 96 well plate | 384 clear plate/ | 384 black plate/ | |
|---|---|---|---|---|
| | Corning # 3696 | Corning # 3701 | Greiner # 788091 | |
| Pre-fill | 30 μl | 15 μl | 2.5 | μl |
| Medium | | | | |
| Cell suspension | 90 μl | 45 μl | 15 | μl |
| Drug | 5 μl (25X) | 2.5 μl (25X) | 2.5 μl (8X) | |
| Oil | 30 μl | 15 μl | 7 | μl |

TABLE 3
Patient Characteristics

| Number of Patients | 44 |
|---|---|
| Age (mean) | 65.1 years |
| Gender | 29 female |
| Tumor Types | |
| Breast | 16 |
| Non-small Cell Lung | 6 |
| Non-Hodgkin's Lymphoma | 4 |
| Pancreas | 3 |
| Ovary | 2 |
| Skin | 3 |
| Other | 10 |
| Performance Status (ECOG mean) | 1.3 |
| Line of Therapy | |
| Adjuvant | 4 |
| 1^{st} | 16 |
| 2^{nd} | 9 |
| 3^{rd} | 5 |
| 4^{th} | 1 |
| 5^{th} or higher | 5 |

TABLE 4
Patterns of MiCK Assay Use

| | |
|---|---|
| Physician Used MiCK Assay | 28 |
| Used only the assay results | 18 |
| Used the assay and other data | 8 |
| Used assay plus other drugs | 9 |
| Used the assay but modified due to organ function | 2 |
| Physician did not use the MiCK assay results | 16 |
| Patient preferred not to use drugs | 7 |
| Patient put on clinical trial | 1 |
| Physician just didn't use results | 8 |

TABLE 5
Correlation of Response with MiCK Assay Use

| | CR | PR | Stable | Progression |
|---|---|---|---|---|
| Physician used assay results | 3 | 8 | 8 | 6 |
| Physician did not use assay results | 0 | 1 | 3 | 11 |

TABLE 6
Comparison of Generic Multi-Source Drugs with Proprietary Single
Source Drugs in the MiCK Drug-Induced Apoptosis Assay.

| | | Generic Drug | Generic Drug | Proprietary |
|---|---|---|---|---|
| | Number | Apoptosis Better | Apoptosis Equal | Drug Apoptosis |
| | of | Than Proprietary | To Proprietary | Better Than |
| Disease | Assays | Drug | Drug | Generic Drug |
| Colon | 7 | 57% | 14% | 29% |
| Breast | 43 | 84% | 14% | 2% |
| Non- | 32 | 78% | 16% | 6% |
| Small | | | | |
| Cell | | | | |
| Lung | | | | |

TABLE 7
Patient characteristics (n = 72)

| Age | 56 years (median) |
|---|---|
| Assay on tumor metastasis | 54% No |
| | 46% Yes |
| Assay on metastatic nodes | 69% No |
| | 31% Yes |
| Assay on primary tumor | 78% No |
| | 22% Yes |
| Site of metastasis | 33% Lymph node |
| | 18% None |
| | 16% Other |
| | 15% Pleural effusion |
| | 12% Liver |
| | 6% Chest wall |

(N=67 tissue samples from breast cancer patients were analyzed with the MiCK assay. Patient characteristics are shown below)

TABLE 8
KU Summary Statistics for Various Drugs
Only drugs where there were at least 9 samples were considered.

| Drug | N | Mean | Median | Std Dev | % >1 | % >3 |
|---|---|---|---|---|---|---|
| 5FU | 29 | 0.7 | 0.6 | 0.65 | 31% | 0% |
| 5FU/Methotrexate | 10 | 1.1 | 1.0 | 0.91 | 40% | 10% |
| Abraxane | 13 | 1.2 | 1.0 | 0.73 | 46% | 0% |
| Carbo | 39 | 1.6 | 1.6 | 1.08 | 67% | 13% |
| Carbo/Taxol | 13 | 3.3 | 3.1 | 1.83 | 92% | 54% |
| Carbo/Taxotere | 13 | 2.6 | 2.4 | 1.55 | 85% | 38% |
| Cisplatin | 36 | 2.2 | 2.3 | 1.47 | 78% | 22% |
| Cytoxan | 39 | 2.8 | 2.6 | 2.07 | 85% | 31% |
| Cytoxan/Doxo | 13 | 3.5 | 3.2 | 1.85 | 92% | 54% |
| Cytoxan/Epi | 11 | 3.2 | 3.4 | 1.43 | 100% | 55% |
| Cytoxan/Taxol | 10 | 2.6 | 2.7 | 1.62 | 80% | 50% |
| Cytoxan/Taxotere | 9 | 4.3 | 4.1 | 2.33 | 100% | 67% |
| Doxil | 14 | 1.1 | 1.1 | 0.63 | 64% | 0% |
| Doxo | 38 | 1.9 | 1.6 | 0.89 | 84% | 11% |
| Epi | 54 | 2.5 | 2.1 | 1.31 | 94% | 22% |
| Eribulin | 11 | 1.0 | 1.0 | 0.54 | 45% | 0% |
| Etoposide | 22 | 1.3 | 1.3 | 0.92 | 55% | 5% |
| Gemzar | 40 | 1.0 | 0.8 | 0.91 | 43% | 3% |
| Ifosfamide | 11 | 1.7 | 1.5 | 1.42 | 64% | 27% |
| Ixabepilone | 23 | 1.3 | 1.2 | 0.84 | 65% | 4% |
| Methotrexate | 30 | 0.9 | 0.9 | 0.60 | 33% | 0% |
| Mitox | 22 | 1.2 | 1.2 | 0.81 | 64% | 0% |
| Oxali | 11 | 1.9 | 1.8 | 1.10 | 82% | 9% |
| Taxol | 41 | 2.1 | 1.9 | 1.78 | 71% | 15% |
| Taxotere | 43 | 2.1 | 1.9 | 1.35 | 77% | 26% |
| Vincristine | 12 | 1.1 | 1.0 | 0.76 | 50% | 0% |
| Vinor | 42 | 1.8 | 1.5 | 1.55 | 64% | 14% |
| Vinor/Xeloda | 10 | 2.1 | 1.6 | 1.69 | 80% | 20.0% |
| Vnbl | 10 | 1.8 | 1.5 | 1.08 | 80% | 10.0% |
| Xeloda | 19 | 0.7 | 0.7 | 0.68 | 21% | 0.0% |

In the following Tables 9-15, to compare two drugs, their KU values were analyzed on a patient level using a paired t-test approach.

TABLE 9
Patient pairwise comparisons of KU: Epirubicin
vs doxorubicin vs mitoxantrone

| | | | Statistical |
|---|---|---|---|
| Drug Compare | Mean Difference (95% CI) | | Significance |
| Epi - Doxo (n = 34) | 0.37 | (0.08 to 0.66) | 0.01 |
| Epi - Mitox (n = 21) | 0.83 | (0.38 to 1.28) | <0.01 |
| Doxo - Mitox (n = 18) | 0.63 | (0.11 to 1.15) | 0.02 |

(These drugs appear to differ from each other with the biggest difference being between Epi and Mitox.)

TABLE 10
Patient pairwise comparisons of KU: Cytoxan vs ifosphamide

| | | Statistical |
|---|---|---|
| Drug Compare | Mean Difference (95% CI) | Significance |
| Cytoxan - Ifosphamide | 0.34 (−0.07 to 0.76) | 0.10 |
| (n = 11) | | |

(There is borderline statistical significance between Cytoxan and Ifosphamide.)

TABLE 11
Patient pairwise comparisons of KU: Carboplatin
vs cisplatin vs oxaliplatin

| | | | Statistical |
|---|---|---|---|
| Drug Compare | Mean Difference (95% CI) | | Significance |
| Cisplatin - Carbo (n = 24) | 0.88 | (0.37 to 1.39) | <0.01 |
| Oxali - Carbo (n = 11) | 0.34 | (−0.14 to 0.82) | 0.15 |
| Cisplatin - Oxali (n = 10) | 0.33 | (−0.07 to 0.73) | 0.09 |

(Cisplatin is statistically higher than Carbo (p < 0.01). It is borderline statistically higher than Oxali (p = 0.09).)

TABLE 12
Patient pairwise comparisons of KU: Vinblastine
vs vincristine vs vinorelbine

| | | | Statistical |
|---|---|---|---|
| Drug Compare | Mean Difference (95% CI) | | Significance |
| Vnbl - Vincristine (n = 7) | 0.14 | (−0.26 to 0.54) | 0.43 |
| Vinor - Vincristine (n = 11) | 0.63 | (0.10 to 1.16) | 0.02 |
| Vinor - Vnbl (n = 10) | 0.14 | (−0.20 to 0.49) | 0.37 |

(The only statistically significant difference is vinorelbine is higher on average than vincristine (p = 0.02).)

TABLE 13
Patient pairwise comparisons of KU: Taxol vs taxotere vs abraxane

| | | | Statistical |
|---|---|---|---|
| Drug Compare | Mean Difference (95% CI) | | Significance |
| Taxotere - Taxol (n = 35) | 0.05 | (−0.54 to 0.65) | 0.85 |
| Taxotere - Abraxane (n = 12) | 0.98 | (0.26 to 1.69) | 0.01 |
| Taxol - Abraxane (n = 12) | 1.20 | (0.26 to 2.14) | 0.02 |

(Both Taxol and Taxotere are statistically significantly larger than Abraxane.)

TABLE 14
Patient pairwise comparisons of KU: Doxil vs doxorubicin

| | | Statistical |
|---|---|---|
| Drug Compare | Mean Difference (95% CI) | Significance |
| Doxo - Doxil (n = 9) | 0.56 (−0.07 to 1.18) | 0.08 |

(The difference between doxil and doxorubicin is borderline statistically significant (p = 0.08).)

TABLE 15
Patient pairwise comparisons of KU: Xeloda vs 5fu:

| | | Statistical |
|---|---|---|
| Drug Compare | Mean Difference (95% CI) | Significance |
| Xeloda - 5FU (n = 13) | 0.26 (−0.26 to 0.77) | 0.30 |

(There is insufficient statistical evidence to conclude a difference between Xeloda and 5FU.)

TABLE 16
For single drugs, in how many cases was the best generic more
effective than the best proprietary in BREAST cancer specimens.

| Condition | Count | |
|---|---|---|
| best generic > best proprietary by more than | 36/43 | (84%) |
| 0.57 and best generic > 1.0 | | |
| how many = (within +/− 0.57) | 6/43 | (14%) |
| best proprietary > best generic by more than | 1/43 | (2%) |
| 0.57 and best proprietary > 1.0 | | |
| how many were all KU < 1.0 | 0/67 | (0%) |

TABLE 17
Comparison of Cytox versus Ifos

| Condition | Count | |
|---|---|---|
| Cytox > Ifosf by more than 0.57 and Cytox > 1 | 2/11 | (18%) |
| Cytox = Ifosf +/− 0.57 and both > 1 | 6/11 | (55%) |
| Ifosf > Cytox + 0.57 | 0/11 | (0%) |
| Cytox and Ifosf both < 1 | 3/11 | (27%) |

TABLE 18
Comparison of Carbo versus Cisplat

| Condition | Count | |
|---|---|---|
| Carbo > Cisplatin by more than 0.57 and Carbo > 1 | 2/24 | (8%) |
| Carbo = Cisplatin +/− 0.57 and both > 1 | 4/24 | (17%) |
| Cisplatin > Carbo + 0.57 | 14/24 | (58%) |
| Cisplatin and Carbo both < 1 | 4/24 | (17%) |

TABLE 19
Comparison of Carbo or Cisplat versus Oxali

| Condition | Count | |
|---|---|---|
| Max (Carbo or Cisplatin) > Oxali by more than 0.57 and | 4/11 | (36%) |
| Max (Carbo or Cisplatin) > 1 | | |
| Max (Carbo or Cisplatin) = Oxali +/− 0.57 and both > 1 | 4/11 | (36%) |
| Oxali > Max (Carbo or Cisplatin) + 0.57 | 1/11 | (9%) |
| Carbo and Cisplatin and Oxali < 1 | 1/11 | (9%) |

TABLE 20
Comparison of Vinroel (Vinor) versus Vincristine (Vcr) and Vnbl

| Condition | Count | |
|---|---|---|
| Vinor > Max (Vcr or Vnbl) by more than 0.57 | 4/14 | (29%) |
| and Vinroel > 1 | | |
| Vinor = Max (Vcr or Vnbl) +/− 0.57 | 5/14 | (36%) |
| and both > 1 | | |
| Max (Vcr or Vnbl) > Vinor + 0.57 | 0/14 | (0%) |
| Vcr and Vnbl and Vinor < 1 | 2/14 | (14%) |

TABLE 21
Comparison of Abraxane versus Taxol and Taxotere

| Condition | Count | |
|---|---|---|
| Abraxane > Max (Taxol, taxotere) by more than 0.57 | 0/13 | (0%) |
| and Abraxane > 1 | | |
| Abraxane = Max (Taxol, taxotere) +/− 0.57 | 2/13 | (15%) |
| and both > 1 | | |
| Max (Taxol, taxotere) > Abraxane + 0.57 | 10/13 | (77%) |
| Abraxane and Taxol and Taxotere < 1 | 1/13 | (8%) |

TABLE 22
Comparison of Taxotere versus Taxol

| Condition | Count | |
|---|---|---|
| Taxotere > Taxol by more than 0.57 | 13/35 | (37%) |
| and Taxotere > 1 | | |
| Taxotere = Taxol +/− 0.57 | 6/35 | (17%) |
| and both > 1 | | |
| Taxol > Taxotere + 0.57 | 11/35 | (31%) |
| Taxol and Taxotere < 1 | 5/35 | (14%) |

TABLE 23
Comparison of Doxil versus Doxo

| Condition | Count |
|---|---|
| Doxil > Doxo by more than 0.57 and Doxil > 1 | 0/9 (0%) |
| Doxil = Doxo +/− 0.57 and both > 1 | 2/9 (22%) |
| Doxo > Doxil + 0.57 | 4/9 (44%) |
| Doxo and Doxil < 1 | 2/9 (22%) |

TABLE 24
Comparison of Xeloda versus 5fu

| Condition | Count |
|---|---|
| Xeloda > 5fu by more than 0.57 and Xeloda > 1 | 2/13 (15%) |
| Xeloda = 5fu +/− 0.57 and both > 1 | 0/13 (0%) |
| 5fu > Xeloda + 0.57 | 2/13 (15%) |
| 5fu and Xeloda < 1 | 8/13 (62%) |

TABLE 25
Comparison of Epirubicin versus Doxorubicin

| Condition | Count |
|---|---|
| Epi > Doxo by more than 0.57 and Epi > 1 | 6/34 (18%) |
| Epi = Doxo +/− 0.57 and both > 1 | 22/34 (65%) |
| Doxo > Epi + 0.57 | 3/34 (9%) |
| Doxo and Epi < 1 | 1/34 (3%) |

TABLE 26
For combinations of drugs, in how many cases was
5fu/metho > 5fu and metho and > 1.0; 5fu/metho =
5fu or metho; 5fu or metho > 5fu/metho; all < 1.0

| Condition | Count |
|---|---|
| 5fu/metho > Max(5fu, Metho) by more than 0.57 | 2/10 (20%) |
| and 5fu/Metho > 1 | |
| 5fu/metho = Max(5fu, Metho) +/− 0.57 | 2/10 (20%) |
| and both > 1 | |
| Max(5fu, Metho) > 5fu/metho + 0.57 | 1/10 (10%) |
| 5fu/metho, 5fu, and metho all < 1 | 4/10 (40%) |

TABLE 27
For combinations of drugs, in how many cases was carbo/taxol >
carbo and taxol and > 1.0; c/t = c or t; c or t > c/t; all < 1.0

| Condition | Count |
|---|---|
| Carbo/taxol > Max(carbo, taxol) by more than 0.57 | 4/12 (33%) |
| and Carbo/taxol > 1 | |
| Carbo/taxol = Max(carbo, taxol) +/− 0.57 | 6/12 (50%) |
| and both > 1 | |
| Max(carbo, taxol) > Carbo/taxol + 0.57 | 1/12 (8%) |
| Carbo, taxol, carbo/taxol all < 1 | 1/12 (8%) |

TABLE 28
For combinations of drugs, in how many cases was
carbo/taxotere > carbo and taxotere and > 1.0; c/taxotere =
c or taxotere; c or taotere > c/taxotere; all < 1.0

| Condition | Count |
|---|---|
| Carbo/taxotere > Max(carbo, taxotere) by more than 0.57 | 2/13 (15%) |
| and Carbo/taxotere > 1 | |
| Carbo/taxotere = Max(carbo, taxotere) +/− 0.57 | 5/13 (38%) |
| and both > 1 | |
| Max(carbo, taxotere) > Carbo/taxotere + 0.57 | 5/13 (38%) |
| Carbo, taxotere, carbo/taxotere all < 1 | 1/13 (8%) |

TABLE 29
For combinations of drugs, in how many cases was
cytox/doxo > cytox and doxo and > 1.0; cytox/doxo =
cytox or doxo; cytox or doxo > cytox/doxo; all < 1.0

| Condition | Count |
|---|---|
| Cytox/doxol > Max(cytox, doxo) by more than 0.57 | 3/12 (25%) |
| and Cytox/doxol > 1 | |
| Cytox/doxol = Max(cytox, doxo) +/− 0.57 | 3/12 (25%) |
| and both > 1 | |
| Max(cytox, doxol) > Cytox/doxol + 0.57 | 5/12 (42%) |
| Cytox, doxol, cytox/doxol all < 1 | 1/12 (8%) |

TABLE 30
For combinations of drugs, in how many cases was
cytox/epi > cyto and epi and > 1.0; cytox/epi =
cytox or epi; cytox or epi > cytox/epi; all < 1.0

| Condition | Count |
|---|---|
| Cytox/epi > Max(cytox, epi) by more than 0.57 | 4/11 (36%) |
| and Cytox/epi > 1 | |
| Cytox/epi = Max(cytox, epi) +/− 0.57 | 3/11 (27%) |
| and both > 1 | |
| Max(cytox, epi) > Cytox/epi + 0.57 | 4/11 (36%) |
| Cytox, epi, cytox/epi all < 1 | 0/11 (0%) |

TABLE 31
For combinations of drugs, in how many cases was
cytox/taxol > cytox and taxol and > 1.0; cytox/taxol =
cytox or taxol; cytox or taxol > cytox/taxol; all < 1.0

| Condition | Count |
|---|---|
| Cytox/taxol > Max(cytox, taxol) by more than 0.57 | 2/10 (20%) |
| and Cytox/taxol > 1 | |
| Cytox/taxol = Max(cytox, taxol) +/− 0.57 | 2/10 (20%) |
| and both > 1 | |
| Max(cytox, taxol) > Cytox/taxol + 0.57 | 6/10 (60%) |
| Cytox, taxol, cytox/taxol all < 1 | 0/10 (0%) |

TABLE 32
For combinations of drugs, in how many cases was
cytox/taxotere > cytox and taxotere and > 1.0; cytox/taxotere =
cyto or taxotere; cytox or taxotere > cytox/taxotere; all < 1.0

| Condition | Count |
|---|---|
| Cytox/taxotere > Max(cytox, taxotere) by more than 0.57 | 3/9 (33%) |
| and Cytox/taxotere > 1 | |
| Cytox/taxotere = Max(cytox, taxotere) +/− 0.57 | 2/9 (22%) |
| and both > 1 | |
| Max(cytox, taxotere) > Cytox/taxotere + 0.57 | 4/9 (44%) |
| Cytox, taxotere, cytox/taxotere all < 1 | 0/9 (0%) |

TABLE 33
For combinations of drugs, in how many cases was vinor/xelo >
vinor and xelo and > 1.0; vinor/xelo = vinor
or xelo; vinor or xelo > vinor/xelo; all < 1.0

| Condition | Count |
|---|---|
| Vinor/xelo > Max(vinor, xelo) by more than 0.57 | 0/10 (0%) |
| and Vinor/xelo > 1 | |
| Vinor/xelo = Max(vinor, xelo) +/− 0.57 | 4/10 (40%) |
| and both > 1 | |
| Max(vinor, xelo) > Vinor/xelo + 0.57 | 4/10 (40%) |
| Vinor, xelo, and vinor/xelo all < 1 | 2/10 (20%) |

TABLE 34
In how many cases was the best generic more effective
than the best proprietary in LUNG cancer Specimens.

| Condition | Count |
|---|---|
| best generic > best proprietary by more than | 25/32 (78%) |
| 0.57 and best generic > 1.0 | |
| how many = (within +/− 0.57) | 5/32 (16%) |
| best proprietary > best generic by more than | 2/32 (6%) |
| 0.57 and best proprietary > 1.0 | |
| how many were all KU < 1.0 | 0/41 (0%) |

TABLE 35
In how many cases was the best generic more effective
than the best proprietary in COLON cancer Specimens.

| Condition | Count |
|---|---|
| best generic > best proprietary by more than | 4/7 (57%) |
| 0.57 and best generic > 1.0 | |
| how many = (within +/− 0.57) | 1/7 (14%) |
| best proprietary > best generic by more than | 2/7 (29%) |
| 0.57 and best proprietary > 1.0 | |
| how many were all KU < 1.0 | 0/8 (0%) |

TABLE 36
Drug Cost Savings from Generic Multi-Source Drug Use Versus Proprietary
Single Source Drug Use Based on the MiCK Drug-Induced Apoptosis Assay.

| | | Proportion of | | | |
|---|---|---|---|---|---|
| | Drug | Patients with | Assay-Adjusted | Net Drug | |
| | Savings | Generic Drug | Drug Savings | Savings | |
| | (Mean) Per | Superiority or | (Mean) Per | (Mean) Per | Percent |
| Disease | Patient | Equivalence | Patient | Patient | Cost Savings |
| Colon | $35,668 | 71% | $25,338 | $20,338 | 54.0% |
| Breast | $13,593 | 98% | $13,321 | $8,321 | 42.8% |
| Non-Small | $15,774 | 94% | $14,827 | $9,827 | 47.0% |
| Cell Lung | | | | | |

TABLE 37
Drug Cost Savings from Generic Multi-Source Drug Use Versus Proprietary
Single Source Drug Use Based on the MiCK Drug-Induced Apoptosis Assay.

| | PROPRIETARY | | | | | | |
|---|---|---|---|---|---|---|---|
| | SINGLE | | GENERIC MUTLI | | AVG | % | SAVING PER |
| CANCER | SOURCE | PMT/6 | SOURCE | PMT/6 | SAVING | MSD | PT |
| BREAST | NAB-PACLI | $26704 | VINOR | $2242 | | | |
| | GEMCIT | $12609 | EPI/CTX | $1355 | | | |
| | CAPECIT | $18976 | CTX/DOCET | $13913 | | | |
| | AVERAGE | $19430 | | $5837 | $13593 | 98% | $8321/PT |
| COLON | FOLFOX | $37670 | FOLFIRI | $1982 | $35688 | 71% | $20338/PT |
| NSCLC | PEM/CIS | $29217 | CARBO/PACLI | $806 | | | |
| | GEM/CIS | $12609 | VINOR | $1601 | | | |
| | | | DOCET | $13009 | | | |
| | AVERAGE | $20913 | | $5138 | $15774 | 94% | $9827/PT |

TABLE 38
Apoptotic response of cancer cells to the 37 tested anti-
cancer drug candidates at various concentrations.

| Drug Tested | Max Resp. (KU) | Resp. Level |
|---|---|---|
| Cisplatin | >10.0 | Sensitive |
| 4HC (cytoxan) | 8.4 | |
| Sunifinib | 7.9 | |
| Oxaliplatin | 6.7 | |
| Carboplatin | 6.0 | |
| Melphalan | 5.3 | |
| Vidaza | 4.3 | Moderate |
| Dactinomycin | 4.0 | |
| Velcade | 3.8 | |
| Sorafenib | 3.8 | |
| Epirubicin | 3.8 | |
| Doxorubicin | 3.5 | |
| 4HI(ifosfamide) + | 3.2 | |
| Epirubicin | | |
| Danorubicin | 3.1 | |
| Vincrelbine | 3.1 | |
| Irinotecan | 2.6 | Low to |
| 4HI(ifosfamide) | 2.6 | Moderate |
| 4HI(ifosfamide) + | 2.5 | |
| Doxorubicin + | | |
| Dacarbazine | | |
| Gemcitabine + | 2.4 | |
| Taxolere | | |
| Taxolere | 2.3 | |
| Methotrexate + | 2.2 | |
| Vinblastine | | |
| Taxol | 1.6 | Low |
| Temozolomide | 1.5 | |
| Gleevec (imatinib) | 1.5 | |
| Procarbazine | 1.3 | |
| Vinblastine | 1.2 | |
| Doxil | 1.2 | |
| Bleomycin | 1.1 | |
| Vincristine | 0.9 | Nonsensitive |
| CCNU | 0.8 | |
| Etoposide | 0.8 | |
| Gemcitabine | 0.8 | |
| Methotrexate | 0.8 | |
| Tarceva | 0.7 | |
| Alimta | 0.6 | |
| Dacarbazine | 0.6 | |
| 5-Fluorouracil | 0.3 | |

What is claimed is:

1. A method of evaluating the relative apoptosis-inducing activity of an anti-cancer drug candidate, comprising:
a) obtaining cancer cells from a tumor specimen;
b) mincing, digesting, and filtering the specimen;
c) optionally removing non-viable cells by density gradient centrifugation;
d) incubating the cell suspension to remove macrophages by adherence;
e) performing positive, negative, and/or depletion isolation to isolate the cells of interest;
f) removing any remaining macrophages, if necessary, using CD14 antibody conjugated magnetic beads;
g) plating the final suspension;
h) incubating the plate;
i) exposing at least one well of a plated final suspension to at least one first anti-cancer drug candidate or mixtures of the first candidate and other substances;
j) exposing at least one well of a plated final suspension to at least one second anti-cancer drug candidate or mixtures of the second candidate and other substances;
k) measuring the optical density of the wells exposed to the at least one first and second anti-cancer drug candidates, or wells containing mixtures of at least one first or at least one second anti-cancer drug candidate and other substances, wherein said measuring of the optical density occurs in a serial manner at selected time intervals for a selected duration of time;
l) determining a kinetic units value for the at least one first and second anti-cancer drug candidates from the optical density and time measurements;
m) correlating the kinetic units value for each drug candidate with:
a) an ability of the anti-cancer drug candidate to induce apoptosis in the cancer cells if the kinetic units value is greater than a predetermined threshold;
b) an inability of the anti-cancer drug candidate to induce apoptosis in the cancer cells if the kinetic units value is less than a predetermined threshold;
n) comparing the determined kinetics units value for each drug candidate; and
o) determining a drug candidate that has a greater relative ability to induce apoptosis in a cancer cell based upon the comparison in step (n).

2. The method of claim 1, wherein the at least one first and second anti-cancer drug candidates comprise at least one generic drug candidate and one proprietary drug candidate.

3. The method of claim 2, further comprising the step of:
p) determining the monetary consequences resultant from choosing either the generic or proprietary drug candidate, wherein the drug candidate with the highest relative kinetic units value is selected.

4. The method of claim 3, wherein the monetary consequences are determined based upon treating a single patient with the selected drug with the higher kinetic units value versus the cost that would have occurred based upon the drug candidate with the lower kinetic units value.

5. The method of claim 3, further comprising the step of:
q) extrapolating the monetary consequences determined from step q) to a target population.

6. The method of claim 5, wherein the target population is a nationwide population from the United States.

7. The method of claim 3, wherein the monetary consequences of step p) are determined by a method comprising:
i) obtaining Medicare cost payment schedules for the selected anti-cancer drug with the higher kinetic units value and also for the drug with the lower kinetic units value;
ii) determining the relative monetary cost savings or relative monetary expenditure that would accrue to a single patient based upon treating said patient with the drug candidate with the higher relative kinetic units value versus treating said patient with the drug candidate with the lower kinetic units value, wherein said treatment comprises at least one cycle of treatment with the selected anti-cancer drug candidate; and
iii) extrapolating the cost savings or relative monetary expenditure from step ii) out to a target population of interest.

8. The method of claim 1, wherein the tumor specimen is a solid tumor specimen, or a blood specimen, or a bone marrow specimen, or an effusion derived specimen.

9. The method of claim 1, wherein at least one of the first or second anti-cancer drug candidates is a combination comprising said anti-cancer drug candidate and at least one additional anti-cancer drug candidate.

10. The method of claim 1, wherein each well of the plate comprises a different anti-cancer drug candidate.

11. The method of claim 1, wherein each well of the plate comprises a different concentration of the anti-cancer drug candidate.

12. The method of claim 1, wherein the anti-cancer drug candidate concentration is from 0.01 to 10,000 μM.

13. The method of claim 1, wherein the optical density is serially measured and recorded approximately every 5 minutes for a period of approximately 48 hours.

14. The method of claim 1, wherein the optical density is measured by a spectrophotometer at a wavelength of from 550 to 650 nanometers.

15. The method of claim 1, wherein the at least one anti-cancer drug candidates are selected from the group consisting of: Abraxane, Alimta, Amsacrine, Asparaginase, Bendamustine, Bleomycin, Bosutinib, Caelyx (Doxil), Carboplatin, Carmustine, CCNU, Chlorambucil, Cisplatin, Cladribine, Clofarabine, Cytarabine, Cytoxan (4HC), Dacarbazine, Dactinomycin, Dasatinib, Daunorubicin, Decitabine, Dexamethasone, Docetaxel, Doxorubicin, Epirubicin, Eribulin, Erlotinib, Estramustine, Etoposide, Everolimus, Fludarabine, 5-Fluorouracil, Gemcitabine, Gleevec (imatinib), Hydroxyurea, Idarubicin, Ifosfamide (4HI), Interferon-2a, Irinotecan, Ixabepilone, Melphalan, Mercaptopurine, Methotrexate, Mitomycin, Mitoxantrone, Nilotinib, Nitrogen Mustard, Oxaliplatin, Paclitaxel, Pentostatin, Procarbazine, Regorafenib, Sorafenib, Streptozocin, Sunitinib, Temozolomide, Temsirolimus, Teniposide, Thalidomide, Thioguanine, Topotecan, Velcade, Vidaza, Vinblastine, Vincristine, Vinorelbine, Vorinostat, Everolimus, Lapatinib, Lenalidomide, Rapamycin, and Votrient (Pazopanib).

16. The method of claim 2, wherein the at least one anti-cancer generic drug candidates are selected from the group consisting of: cyclophosphamide, doxorubicin, epirubicin, paclitaxel, docetaxel, cisplatin, carboplatin, irinotecan, topotecan, vinorelbine, and vinblastine.

17. The method of claim 2, wherein the at least one anti-cancer proprietary drug candidates are selected from the group consisting of: nab-paclitaxel, gemcitabine, oxaliplatin, capcitabine, ixabepilone, erubilin, liposomal doxorubicin, and pemetrexed.

18. A method of tumor cell isolation and purification, comprising:
a) obtaining a tumor specimen;
b) mincing, digesting, and filtering the specimen;
c) optionally removing non-viable cells by density gradient centrifugation;
d) incubating the cell suspension to remove macrophages by adherence;
e) performing positive, negative, and/or depletion isolation to isolate the cells of interest;
f) removing any remaining macrophages, if necessary, using CD14 antibody conjugated magnetic beads;
g) plating the final suspension; and
h) incubating the plate.

19. A method of evaluating the ability of an anti-cancer drug candidate to induce apoptosis in a cancer cell line derived from a tumor specimen, comprising:
a) obtaining a tumor specimen;
b) mincing, digesting, and filtering the specimen;
c) optionally removing non-viable cells by density gradient centrifugation;
d) incubating the cell suspension to remove macrophages by adherence;
e) performing positive, negative, and/or depletion isolation to isolate the cells of interest;
f) removing any remaining macrophages, if necessary, using CD14 antibody conjugated magnetic beads;
g) plating the final suspension;
h) incubating the plate;
i) exposing at least one well of a plated final suspension to at least one anti-cancer drug candidate or mixtures of the candidate and other substances;
j) measuring the optical density of the wells exposed to the at least one anti-cancer drug candidate, or wells containing mixtures of at least one anti-cancer drug candidate and other substances, wherein said measuring of the optical density occurs in a serial manner at selected time intervals for a selected duration of time;
k) determining a kinetic units value for the at least one anti-cancer drug candidate from the optical density and time measurements; and
l) correlating the kinetic units value for each drug candidate with:
a) an ability of the anti-cancer drug candidate to induce apoptosis in the cancer cells if the kinetic units value is greater than a predetermined threshold;
b) an inability of the anti-cancer drug candidate to induce apoptosis in the cancer cells if the kinetic units value is less than a predetermined threshold.

20. The method of claim 19, wherein each well of the plate comprises a different anti-cancer drug candidate.

21. The method of claim 19, wherein each well of the plate comprises a different concentration of the anti-cancer drug candidate.

22. The method of claim 19, wherein the anti-cancer drug candidate concentration is from 0.01 to 10,000 μM.

23. The method of claim 19, wherein the optical density is serially measured and recorded approximately every 5 minutes for a period of approximately 48 hours.

24. The method of claim 19, wherein the optical density is measured by a spectrophotometer at a wavelength of from 550 to 650 nanometers.

25. The method of claim 19, wherein the tumor specimen is a solid tumor specimen, or a blood specimen, or a bone marrow specimen, or an effusion derived specimen.

26. The method of claim 19, wherein the anti-cancer drug candidates are selected from the group consisting of: Abraxane, Alimta, Amsacrine, Asparaginase, Bendamustine, Bleomycin, Bosutinib, Caelyx (Doxil), Carboplatin, Carmustine, CCNU, Chlorambucil, Cisplatin, Cladribine, Clofarabine, Cytarabine, Cytoxan (4HC), Dacarbazine, Dactinomycin, Dasatinib, Daunorubicin, Decitabine, Dexamethasone, Docetaxel, Doxorubicin, Epirubicin, Eribulin, Erlotinib, Estramustine, Etoposide, Everolimus, Fludarabine, 5-Fluorouracil, Gemcitabine, Gleevec (imatinib), Hydroxyurea, Idarubicin, Ifosfamide (4HI), Interferon-2a, Irinotecan, Ixabepilone, Melphalan, Mercaptopurine, Methotrexate, Mitomycin, Mitoxantrone, Nilotinib, Nitrogen Mustard, Oxaliplatin, Paclitaxel, Pentostatin, Procarbazine, Regorafenib, Sorafenib, Streptozocin, Sunitinib, Temozolomide, Temsirolimus, Teniposide, Thalidomide, Thioguanine, Topotecan, Velcade, Vidaza, Vinblastine, Vincristine, Vinorelbine, Vorinostat, Everolimus, Lapatinib, Lenalidomide, Rapamycin, and Votrient (Pazopanib).
